(19) United States
(12) Patent Application Publication
(10) Pub. No.: US 2024/0011047 A1
(43) Pub. Date: Jan. 11, 2024

(54) CMV VECTORS AND USES THEREOF

(71) Applicants: The Regents of the University of California, Oakland, CA (US); The UAB Research Foundation, Birmingham, AL (US)

(72) Inventors: Peter Barry, Davis, CA (US); Dennis Hartigan-O'Connor, Kensington, CA (US); Ellen Sparger, Davis, CA (US); William Chang, Sacramento, CA (US); Mark Walter, Birmingham, AL (US); Corey Miller, San Francisco, CA (US)

(73) Assignees: The Regents of the University of California, Oakland, CA (US); The UAB Research Foundation, Birmingham, AL (US)

(21) Appl. No.: 18/064,809

(22) Filed: Dec. 12, 2022

Related U.S. Application Data
(63) Continuation of application No. 17/013,077, filed on Sep. 4, 2020, now abandoned.
Application No. 17/013,077 is a continuation of application No. PCT/US2019/021469, filed on Mar. 8, 2019.
(60) Provisional application No. 62/641,175, filed on Mar. 9, 2018.

(51) Int. Cl. C12N 15/86; A61P 31/20; A61P 35/00; A61K 39/00; A61K 39/21; C07K 14/005; C07K 14/705

(52) U.S. Cl. CPC C12N 15/86; A61P 31/20; A61P 35/00; A61K 39/00119; A61K 39/21; C12N 2740/15034; C07K 14/705; A61K 2039/5256; C12N 2710/16122; C12N 2710/16134; C12N 2710/16143; C07K 14/005

(57) ABSTRACT

In one aspect, the present invention provides recombinant polynucleotides. In some embodiments, the recombinant polynucleotides comprise a cytomegalovirus (CMV) genome, or a portion thereof, and a nucleic acid sequence encoding an antigen, wherein the CMV genome or portion thereof comprises a mutation within a interleukin-10-like gene sequence. Methods for preventing and treating diseases such as infectious diseases and cancer are also provided herein.

### CROSS-REFERENCE TO RELATED APPLICATIONS

This application is a continuation of U.S. application Ser. No. 17/013,077, filed Sep. 4, 2020, which is a continuation of International Application No. PCT/US2019/021469, filed Mar. 8, 2019, which claims priority to U.S. Provisional Application No. 62/641,175, filed Mar. 9, 2018, the disclosures of which are herein incorporated by reference in their entirety for all purposes.

### STATEMENT AS TO RIGHTS TO INVENTIONS MADE UNDER FEDERALLY SPONSORED RESEARCH AND DEVELOPMENT

This invention was made with Government support under Grant Nos. AI097629 and AI049342, awarded by the National Institutes of Health. The Government has certain rights in this invention.

### SEQUENCE LISTING

A Sequence Listing conforming to the rules of WIPO Standard ST.26 is hereby incorporated by reference. The Sequence Listing has been filed as an electronic document via EFS-Web in ASCII format encoded as XML. The electronic document, created on Dec. 7, 2022, is entitled “070772-224520US-1361311_ST26.xml”, and is 23,994 bytes in size.

### BACKGROUND OF THE INVENTION

Vaccines provide an effective approach to prevent and treat a large number of diseases, including numerous infectious diseases as well as cancer. Among the various types of vaccines that are available, viral vector vaccines are particularly attractive, as they can produce robust and broad immune responses, including increased cellular immunity, while at the same time being amenable to engineering that reduces or eliminates pathogenicity in the subject being vaccinated. However, viral vectors can also encode proteins that suppress the immune response, thus reducing vaccine effectiveness. Accordingly, there is a need for improved viral vector vaccines that afford enhanced immunogenicity. The present invention satisfies this need, and provides related advantages as well.

### BRIEF SUMMARY OF THE INVENTION

In a first aspect, the present invention provides a recombinant polynucleotide. In some embodiments, the recombinant polynucleotide comprises a cytomegalovirus (CMV) genome, or a portion thereof, and a nucleic acid sequence encoding an antigen, wherein the CMV genome or portion thereof comprises one or more immunomodulatory mutations, wherein the one or more immunomodulatory mutations comprise a mutation within a nucleic acid sequence encoding a protein that has interleukin-10 (IL-10)-like activity. In some embodiments, the CMV is a CMV that can infect human, non-human primate, or mouse cells. In some embodiments, the protein that has IL-10-like activity is human CMV IL-10 (HCMVIL-10) or rhesus macaque CMV IL-10 (RhCMVIL-10).

In some embodiments, the nucleotide sequence encoding the antigen is located within the CMV genome or portion thereof. In some embodiments, the one or more immunomodulatory mutations comprise a substitution, a deletion, and/or an insertion of one or more nucleotides. In some embodiments, the mutation within the nucleic acid sequence encoding the protein that has IL-10-like activity comprises a deletion within or complete deletion of the first two exons of the nucleic acid sequence encoding the protein that has IL-10-activity. In some embodiments, the one or more immunomodulatory mutations are located in a regulatory region and/or a protein coding region of the nucleic acid sequence encoding the protein that has IL-10-like activity. In some embodiments, the mutation within the nucleic acid sequence encoding the protein that has IL-10-like activity reduces or inactivates the activity of the protein having IL-10-like activity.

In some embodiments, the antigen is a non-CMV antigen. In some embodiments, the antigen is an infectious disease antigen. In some embodiments, the infectious disease antigen is a bacterial, viral, fungal, protozoal, and/or helminthic infectious disease antigen. In some embodiments, the infectious disease antigen is a viral infectious disease antigen from simian immunodeficiency virus (SIV), human immunodeficiency virus (HIV), hepatitis C virus, herpes simplex virus, Epstein-Barr virus, or a combination thereof. In some embodiments, the infectious disease antigen comprises an HIV or SIV group-specific antigen (gag) protein. In some embodiments, the infectious disease antigen is a bacterial infectious disease antigen from Mycobacterium tuberculosis.

In some embodiments, the antigen is a tumor-associated antigen. In some embodiments, the tumor-associated antigen is selected from the group consisting of prostate-specific antigen, melanoma-associated antigen 4 (MAGEA4), melanoma-associated antigen 10 (MAGEA10), NY-ESO-1, a neoantigen, and a combination thereof.

In some embodiments, the one or more immunomodulatory mutations further comprise an insertion of a nucleic acid sequence encoding an immunostimulatory protein. In some embodiments, the immunostimulatory protein is a cytokine. In some embodiments, the cytokine is selected from the group consisting of interleukin-12 (IL-12), interleukin-15 (IL-15), and a combination thereof.

In some embodiments, the CMV is a CMV capable of infecting rhesus macaque cells and the one or more immunomodulatory mutations further comprise a mutation within a region of the CMV genome or portion thereof selected from the group consisting of Rh182, Rh183, Rh184, Rh185, Rh186, Rh187, Rh188, Rh189, and a combination thereof. In some embodiments, the CMV is a CMV capable of infecting human cells and the one or more immunomodulatory mutations further comprise a mutation within a region of the CMV genome or portion thereof selected from the group consisting of US2, US3, US4, US5, US6, US7, US8, US9, US10, US11, and a combination thereof.

In some embodiments, the one or more immunomodulatory mutations further comprise a mutation within a nucleic acid sequence encoding a protein that inhibits antigen presentation by a major histocompatibility complex (MHC) molecule. In some embodiments, the CMV genome or portion thereof further comprises a mutation that increases tropism for a target cell. In some embodiments, the target cell is selected from the group consisting of an antigen-presenting cell, a tumor cell, a fibroblast, an epithelial cell, an endothelial cell, and a combination thereof. In some embodiments, the antigen-presenting cell is a dendritic cell. In some embodiments, the mutation that increases tropism comprises a mutation that modifies a protein, or a portion thereof, that is positioned on the outside of a CMV virion. In some embodiments, the mutation that increases tropism comprises an insertion of a nucleotide sequence encoding a cellular targeting ligand.

In some embodiments, the cellular targeting ligand is selected from the group consisting of an antibody fragment that recognizes a target cell antigen, a ligand that is recognized by a target cell cognate receptor, a viral capsid protein that recognizes a target cell, and a combination thereof. In some embodiments, the cellular targeting ligand is CD154.

In some embodiments, the CMV is a CMV capable of infecting rhesus macaque cells and the mutation that increases tropism comprises a mutation within a gene selected from the group consisting of Rh13.1, Rh61/Rh60, Rh157.4, Rh157.5, Rh157.6, and a combination thereof.

In some embodiments, the CMV is a CMV capable of infecting human cells and the mutation that increases tropism comprises a mutation within a gene selected from the group consisting of RL13, UL36, UL130, UL128, UL131, and a combination thereof.

In some embodiments, the one or more immunomodulatory mutations further comprise a mutation that increases or decreases the unfolded protein response (UPR). In some embodiments, the mutation that increases or decreases the UPR decreases or increases the expression of Human cytomegalovirus UL50, Rhesus cytomegalovirus Rh81, or Mouse cytomegalovirus M50.

In some embodiments, the polynucleotide further comprises a nucleic acid sequence encoding a selectable marker. In some embodiments, the nucleic acid sequence encoding the selectable marker is located within the CMV genome or portion thereof. In some embodiments, the nucleic acid sequence encoding the selectable marker comprises a nucleic acid sequence encoding an antibiotic resistance gene and/or a fluorescent protein.

In some embodiments, the recombinant polynucleotide contains one or more regulatory sequences. In some embodiments, the one or more regulatory sequences control the expression of a gene or region within the CMV genome or portion thereof, the antigen-encoding sequence, an immunostimulatory protein-encoding sequence, a selectable marker-encoding sequence, a variant thereof, or a combination thereof. In some embodiments, the one or more regulatory sequences comprise a CMV early enhancer, a chicken beta-actin gene promoter, a first exon of a chicken beta-actin gene, a first intron of a chicken beta-actin gene, a splice acceptor of a rabbit beta-globin gene, an EM7 promoter, an EF1α promoter, or a combination thereof.

In a second aspect, a viral particle is provided. In some embodiments, the viral particle comprises a recombinant polynucleotide of the present invention.

In a third aspect, a host cell is provided. In some embodiments, the host cell comprises a recombinant polynucleotide of the present invention or a viral particle of the present invention.

In another aspect, a pharmaceutical composition is provided. In some embodiments, the pharmaceutical composition comprises a recombinant polynucleotide of the present invention, a viral particle of the present invention, and/or a host cell of the present invention; and a pharmaceutically acceptable carrier.

In yet another aspect, a method for inducing an immune response against an antigen in a subject is provided. In some embodiments, the method comprises administering to the subject a therapeutically effective amount of a pharmaceutical composition of the present invention. In some embodiments, the antigen is an infectious disease antigen or a tumor-associated antigen. In some embodiments, the infectious disease antigen is a bacterial, viral, fungal, protozoal, and/or helminthic infectious disease antigen. In some embodiments, the viral infectious disease antigen is from simian immunodeficiency virus (SIV), human immunodeficiency virus (HIV), hepatitis C virus, herpes simplex virus, Epstein-Barr virus, or a combination thereof. In some embodiments, the infectious disease antigen is a bacterial infectious disease antigen from Mycobacterium tuberculosis. In some embodiments, the tumor-associated antigen is selected from the group consisting of prostate-specific antigen, melanoma-associated antigen 4 (MAGEA4), melanoma-associated antigen 10 (MAGEA10), NY-ESO-1, a neoantigen, and a combination thereof.

In some embodiments, the immune response induced in the subject is greater than the immune response that is induced using a recombinant polynucleotide that does not comprise the mutation within the nucleic acid sequence encoding the protein that has IL-10-like activity. In some embodiments, inducing the immune response comprises generating antibodies that recognize the antigen. In some embodiments, inducing the immune response comprises increasing the expression or activity of interferon-gamma and/or tumor necrosis factor-alpha in the subject. In some embodiments, inducing the immune response comprises increasing the number or activation of MHC-E-restricted T cells in the subject. In some embodiments, the unfolded protein response (UPR) is increased or decreased in the subject.

In some embodiments, a sample is obtained from the subject. In some embodiments, the sample is selected from the group consisting of a blood sample, a tissue sample, a urine sample, a saliva sample, a cerebrospinal fluid (CSF) sample, and a combination thereof. In some embodiments, the level of one or more biomarkers is determined in the sample. In some embodiments, the one or more biomarkers is selected from the group consisting of C-reactive protein, interferon-gamma, IL-4, IL-5, IL-6, IL-10, IL-12, IL-15, tumor necrosis factor-alpha, and a combination thereof.

In some embodiments, the level of the one or more biomarkers is compared to a reference sample. In some embodiments, the reference sample is obtained from the subject. In other embodiments, the reference sample is obtained from a different subject or a population of subjects.

In still another aspect, a method for preventing or treating a disease in a subject is provided. In some embodiments, the method comprises administering to the subject a therapeutically effective amount of a pharmaceutical composition of the present invention. In some embodiments, the disease is an infectious disease or cancer. In some embodiments, the infectious disease is a bacterial, viral, fungal, protozoal, and/or helminthic infectious disease. In some embodiments, the viral infectious disease is caused by a virus selected from the group consisting of simian immunodeficiency virus (SIV), human immunodeficiency virus (HIV), hepatitis C virus, herpes simplex virus, and Epstein-Barr virus. In some embodiments, the cancer is melanoma, ovarian cancer, or prostate cancer. In some embodiments, treating the subject comprises decreasing or eliminating one or more signs or symptoms of the disease.

Other objects, features, and advantages of the present invention will be apparent to one of skill in the art from the following detailed description and figures.

### BRIEF DESCRIPTION OF THE DRAWINGS

FIGS. 1A and 1B depict the construction of viral IL-10-deleted RhCMV vectors. FIG. 1A depicts the removal of the coding capacity of rhesus macaque CMV viral IL-10 (i.e., the rhesus macaque ortholog of the human CMV UL111A open reading frame (ORF)) by Red/ET-mediated recombination of the viral genome with a DNA fragment carrying an EM7-Zeocin resistance (Zeocin^{R}) cassette, as well as upstream and downstream flanking regions. FIG. 1B depicts the resulting RhCMVΔIL-10 genome that carries the EM7-Zeocin^{R} cassette instead of the complete RhCMVIL-10 ORF.

FIG. 2 depicts the construction of a viral IL-10-deleted RhCMV vaccine carrying the SIV gag sequence. The SIV gag cassette was placed between the RhCMV Rh213 and Rh214 ORFs via Red/ET-mediated recombination. Deletion within the viral IL-10 gene is described above and depicted in FIG. 1.

FIG. 3 shows that rhesus macaques immunized with viral IL-10-deleted RhCMV-gag exhibited significantly higher CD4⁺ T cell responses against the vaccine target. * denotes p<0.05. ** denotes p<0.01.

FIG. 4 shows a timeline of a study to test the magnitude and character of immune responses to a viral IL-10-deleted RhCMV/SIV gag vaccine.

FIGS. 5A-5D show the results of experiments examining immune responses to vaccination with the SIV gag antigen. FIGS. 5A and 5B show data that illustrate T cell responses to a RhCMV/SIV gag vaccine. FIGS. 5C and 5D show data that illustrate T cell responses to RhCMV/SIV gag or RhCMVΔIL-10/SIV gag vaccines.

FIGS. 6A-6C show that a RhCMVΔIL-10/SIV gag vaccine has superior function. For all panels, animals were challenged using the same serial, low-dose, oral challenge protocol. Viral loads in plasma are shown on the y axis and the time after infection is shown on the x axis. Time after infection for all animals was synchronized to the time of first detection of virus. FIG. 6A shows that unvaccinated animals (about 10 months old) were unable to control SIV infection. FIG. 6B shows that of twelve animals receiving the conventional RhCMV-based vaccine, only one exhibited control (lower trace labeled “8% control”). FIG. 6C shows that of six animals receiving a viral IL-10-deficient RhCMV-based vaccine (RhCMVΔIL-10/SIV gag), three exhibited stringent control, each attaining a viral load below the limit of detection within weeks (lower traces labeled “50% control”).

FIG. 7 shows that animals protected from SIV by RhCMVdIL10/SIVgag vaccination had no residual circulating virus. Bars show the amount of virus detectable after 18 days of in vitro amplification in the presence of CEMx174 cells, which are highly susceptible to infection. Plasma samples from two animals that were not protected by the vaccine, for example, grew to a titer of >10⁹ copies per mL within 18 days (animal IDs 45918 and 45947). A sample from a partially protected animal with a low viral load (46061) grew to a much lower concentration (580 copies/mL). No virus was grown from plasma taken from animals protected by the vaccine (46025, 46056, and 46057).

FIG. 8 shows that depletion of CD8⁺ cells from animals protected from SIV by RhCMVdIL10/SIVgag vaccination revealed no residual circulating virus. Lines show the amount of virus detected in the plasma of vaccinated animals treated with CD8-depleting antibody. Plasma samples from one animal that was not completely protected by the vaccine, for example, spiked in the days following first administration of anti-CD8 antibody (animal ID 46061; filled circles). No virus was detectable before or after antibody administration in plasma taken from animals protected by the vaccine (46025, 46056, and 46057; open diamonds).

FIG. 9 shows a therapeutic effect of viral IL-10-deficient SIV gag vaccine (RhCMVΔIL-10/SIVgag) after SIV infection. Lines show the amount of virus detected in the plasma of eight animals in the study. Control animals (not vaccinated) are shown using gray traces and filled circles; vaccinated animals are shown using black traces and open diamonds. Note that most animals (5/8) rebounded after removal of triple therapy at day 238. However, two vaccinated animals (50%) maintained immunologic control over the virus, reducing viral load below 100 copies/mL (dashed lines).

FIGS. 10A-10D show construction and verification of RhCMVdIL10-MAGEA4 and -MAGEA10 vaccines. FIG. 10A shows the results of PCR amplification reactions that verified MAGEA4 and MAGEA10 inserts in the bacterial artificial chromosome (BAC) forms of the vaccines. The PCR primers flanked the insertion sites and thus amplified the entire antigen expression cassette. The expected band for MAGEA4 was 3.8 kb and the expected band for MAGEA10 was 3.9 kb. Two clones (1) and (2) were tested for each vaccine. FIG. 10B used the same strategy as demonstrated in FIG. 10A; the amplifications showed that replicating RhCMVdIL10-MAGEA4 and -MAGEA10 viruses recovered from BAC clones retained the MAGEA4 and MAGEA10 cassettes over two passages in tissue culture, labeled “P0” and “P1.” Two clones were again tested for each virus. The amplifications also demonstrated that the MAGE insertions were stable. FIG. 10C shows additional PCR amplifications that demonstrated that the viral IL-10 gene was deleted in both vaccines. Two PCR reactions were performed: at left, a 1-kb band was amplified from the Zeocin-resistance cassette present in place of the viral IL-10 (UL111A) gene's first two exons. The band was successfully amplified from a control viral IL-10-deficient BAC (lane 3) and from RhCMVdIL10-MAGEA4 and -MAGEA10 viruses (lanes 4-7). At right, a 1.5-kb band was found to be amplified from the intact UL111A gene in a control virus (lane 2) but not from the MAGEA4 and MAGEA10 vaccines (lanes 4-7). FIG. 10D shows confirmation of MAGEA4 protein expression from the RhCMVdIL10-MAGEA4 vaccine. Cell lysates were collected at the end of passages 0, 1, and 2 (P0, P1, P2) for RhCMVdIL10-MAGEA4 clones 1 and 2. 30 micrograms of protein per lane were loaded and MAGEA4 protein was detected using a mouse anti-MAGEA4 monoclonal antibody, sheep anti-mouse HRP, and DAB substrate. MAGEA4 protein was detected from both clones at all passages.

### DETAILED DESCRIPTION OF THE INVENTION

### I. Introduction

Cytomegalovirus (CMV) comprises several different viruses that are species-specific members of the herpesvirus family, including rhesus macaque CMV (RhCMV) and human CMV (HCMV). There has been particular interest in developing vaccines based on RhCMV and HCMV vectors, among others. A limiting feature of using CMV-based vectors for vaccination is that the genomes of these viruses encode proteins that facilitate viral persistence in the host by suppressing the host's immune response. The present invention is based, in part, on the discovery that CMV-based vaccine vectors in which part of the nucleic acid sequence encoding a protein that has interleukin-10 (IL-10)-like activity has been deleted produce enhanced immune responses against antigenic proteins encoded by the vector. CMV-based vectors of the present invention are useful for, among other things, preventing and treating a large number of diseases, including various infectious diseases as well as cancer.

### II. Definitions

As used herein, the following terms have the meanings ascribed to them unless specified otherwise.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

The terms “about” and “approximately” as used herein shall generally mean an acceptable degree of error for the quantity measured given the nature or precision of the measurements. Typically, exemplary degrees of error are within 20 percent (%), preferably within 10%, and more preferably within 5% of a given value or range of values. Any reference to “about X” specifically indicates at least the values X, 0.8X, 0.81X, 0.82X, 0.83X, 0.84X, 0.86X, 0.87X, 0.88X, 0.89X, 0.9X, 0.91X, 0.92X, 0.93X, 0.94X, 0.95X, 0.96X, 0.97X, 0.99X, 1.01X, 1.02X, 1.03X, 1.04X, 1.05X, 1.06X, 1.07X, 1.08X, 1.09X, 1.1X, 1.11X, 1.12X, 1.13X, 1.14X, 1.15X, 1.16X, 1.17X, 1.18X, 1.19X, and 1.2X. Thus, “about X” is intended to teach and provide written description support for a claim limitation of, e.g., “0.98X.”

The term “cytomegalovirus” or “CMV” refers to viruses that include members of the Cytomegalovirus genus of viruses (within the order Herpesvirales, family Herpesviridae, subfamily Betaherpesvirinae). The term includes, but is not limited to, Human cytomegalovirus (HCMV; also known as Human herpesvirus 5 (HHV-5)), Simian cytomegalovirus (SCCMV or AGMCMV), Baboon cytomegalovirus (BaCMV), Owl monkey cytomegalovirus (OMCMV), Squirrel monkey cytomegalovirus (SMCMV), and Rhesus cytomegalovirus (RhCMV) that infects macaques.

The term “protein that has interleukin-10-like activity” or “protein that has IL-10-like activity” refers to any protein that functions in a similar way to interleukin-10 (IL-10) or produces a similar effect (e.g., has a similar immunomodulatory effect) to IL-10. The term includes, but is not limited to, proteins encoded by viral IL-10 genes (e.g., CMV IL-10 genes) such as HCMVIL-10 in HCMV and RhCMVIL-10 in RhCMV, proteins that bind to an IL-10 receptor, proteins that stimulate downstream IL-10 receptor signaling, and functional portions thereof. The term also includes, but is not limited to, proteins encoded by corresponding viral IL-10 genes in SCCMV/AGMCMV, BaCMV, OMCMV, and SMCMV, as well as homologs thereof. A protein that has IL-10-like activity can, for example, downregulate the expression of Th1 and macrophage cytokines (e.g., interferon-gamma, IL-1-beta, IL-2, IL-6, IL-12, TNF-alpha, and GM-CSF), MHC class II antigens, and/or macrophage co-stimulatory molecules; promote blockade of NF-κB activity; and/or enhance B cell survival, proliferation, and/or antibody production. Non-limiting examples of nucleic acid sequences that encode proteins having IL-10-like activity are set forth under SEQ ID NOS:11 and 12.

The term “cytokine” refers to a broad category of small proteins, typically between about 5 kDa and about 20 kDa in size, that are typically secreted and that function in cell signaling, typically by binding to cellular receptors that transmit signals to the intracellular environment of target cells. Cytokines include interleukins, chemokines, interferons, lymphokines, monokines, and tumor necrosis factors. Cytokines are produced by immune cells (e.g., monocytes, macrophages, B lymphocytes, T lymphocytes, and mast cells), endothelial cells, fibroblasts, and stromal cells. Cytokines play diverse roles in immune responses, inflammation, and responses to infection, trauma, and sepsis, as well as cancer. In the context of immune function, cytokines regulate, among other things, the balance between humoral immunity and cell-based immunity, as well as the balance between different types of cell-based immunity, e.g., Th1- versus Th2-predominant cell-based immunity. Cytokines also regulate the maturation and growth of immune cells. Cytokines can either increase or decrease an immune response, depending on the particular cytokine.

The term “interleukin” refers to a group of cytokines that play important roles in innate and adaptive immune system function. For example, some interleukins promote the development and differentiation of B lymphocytes, T lymphocytes, and hematopoietic cells. Most interleukins are produced by helper CD4 T lymphocytes, monocytes, macrophages, and endothelial cells. Interleukins can either enhance or inhibit immune function, depending on the particular interleukin.

Examples of interleukins (ILs) include IL-1 (which targets T helper cells, B cells, natural killer (NK) cells, macrophages, and endothelial cells, among others), IL-2 (which targets activated T cells and B cells, regulatory T cells, NK cells, macrophages, and oligodendrocytes), IL-3 (which targets hematopoietic stem cells and mast cells), IL-4 (which targets activated B cells, T cells, and endothelial cells), IL-5 (which targets B cells and eosinophils), IL-6 (which targets activated B cells, plasma cells, hematopoietic cells, and T cells, among others), IL-7 (which targets pre/pro-B and pre/pro-T cells, as well as NK cells), IL-8 (also known as CXCL8, which targets neutrophils, basophils, and lymphocytes), IL-9 (which targets T cells and B cells), IL-10 (which targets macrophages, B cells, mast cells, Th1 cells, and Th2 cells), IL-11 (which targets bone marrow stromal cells), IL-12 (which targets activated T cells and NK cells), IL-13 (which targets Th2 cells, B cells, and macrophages), IL-14 (which targets activated B cells), IL-15 (which targets T cells and activated B cells), IL-16 (which targets CD4+ T cells), IL-17 (which targets epithelial and endothelial cells, among others), IL-18 (which targets Th1 cells and NK cells), IL-19, IL-20, IL-21 (which targets dendritic cells and all lymphocytes), IL-22, IL-23, IL-24, IL-25, IL-26, IL-27, IL-28, IL-29, IL-30, IL-31, IL-32, IL-33, IL-34, IL-35, and IL-36.

“Interleukin-4” or “IL-4” induces differentiation of native helper T cells (Th0 cells) to Th2 cells. Subsequently, upon activation by IL-4, Th2 cells produce additional IL-4 in a positive feedback loop. IL-4 also functions to stimulate proliferation of activated B and T cells, differentiation of B cells into plasma cells, induction of B cell class switching to IgE, and upregulation of MHC class II production. IL-4 also decreases the production of Th1 cells, macrophages, interferon-gamma, and dendritic cell IL-12. Non-limiting examples of human IL-4 amino acid sequences are set forth under NCBI Reference Sequence numbers NP_000580, NP_758858, and NP_001341919.

“Interleukin-5” or “IL-5” is produced by Th2 cells and mast cells, and functions to stimulate B cell growth and increase immunoglobulin secretion. IL-5 is also an important mediator of eosinophil activation. A non-limiting example of a human IL-5 amino acid sequence is set forth under NCBI Reference Sequence number NP_000870.

“Interleukin-6” or “IL-6” is produced by macrophages, Th2 cells, B cells, astrocytes, and endothelial cells. IL-6 acts as a pro-inflammatory cytokine (e.g., in response to infection or tissue damage arising, e.g., from trauma or burns), although it can also act as an anti-inflammatory myokine. Non-limiting examples of human IL-6 amino acid sequences are set forth under NCBI Reference Sequence numbers NP_000591 and NP_001305024.

“Interleukin-10” or “IL-10” is an anti-inflammatory cytokine that is encoded by the IL10 gene in humans. IL-10, which is a homodimer having subunits that are each 178 amino acids in length, binds to a receptor complex that consists of two IL-10 receptor-1 proteins and two IL-10 receptor-2 proteins. Binding of IL-10 to the receptor complex induces STAT3 signaling, via JAK1 phosphorylation of the cytoplasmic tails of IL-10 receptor-1 and Tyk2 phosphorylation of the cytoplasmic tails of IL-10 receptor-2. IL-10 is produced by subsets of monocytes, Th2 cells, CD8⁺ T cells, mast cells, macrophages, and B cells. IL-10 has multiple effects, including but not limited to, downregulation of the expression of Th1 and macrophage cytokines (e.g., interferon-gamma, IL-1-beta, IL-2, IL-6, IL-12, TNF-alpha, and GM-CSF), MHC class II antigens, and/or macrophage co-stimulatory molecules; blockade of NF-κB activity; and/or enhancement of B cell survival, proliferation, and/or antibody production. A non-limiting example of a human IL-10 amino acid sequence is set forth under NCBI Reference Sequence number NP_000563 and SEQ ID NO:14. A non-limiting example of a rhesus macaque IL-10 amino acid sequence is set forth under SEQ ID NO:13.

Many pathogens, including CMV, exploit the IL-10 pathway to enhance pathogen persistence. For example, many CMVs encode their own IL-10 (e.g., HCMVIL-10, also known as UL111, for HCMV and RhCMVIL-10, also known as Rh143, for RhCMV). These viral IL-10 proteins have different amino acid sequences from the human IL-10 protein, but are nonetheless capable of binding IL-10 receptors. Thus, CMV-infected cells will produce viral IL-10, which in turn inhibits the immune response against CMV infection and enhances CMV persistence within the host.

“Interleukin-12” or “IL-12” is produced by dendritic cells, macrophages, neutrophils, and B-lymphoblastoid cells in response to antigenic stimulation. IL-12 is involved in the differentiation of naïve T cells into Th1 cells, and also plays a role in the enhancement of the cytotoxic activity of NK cells and CD8⁺ T cells.

“Interleukin-15” or “IL-15” is secreted by mononuclear phagocytes, among other cells, in response to viral infection and induces the proliferation of NK cells, an important function of which is to kill virally infected cells. Non-limiting examples of human IL-15 amino acid sequences are set forth under NCBI Reference Sequence numbers NP_000576 and NP_751915.

The term “tumor necrosis factor-alpha” or “TNF-alpha” refers to the cytokine that is encoded by the TNFA gene in humans. TNF-alpha is produced by activated macrophages, CD4⁺ T cells, NK cells, neutrophils, eosinophils, mast cells, and neurons. TNF-alpha is involved in processes such as the induction of fever, apoptosis, cachexia, and inflammation, as well as the inhibition of tumorigenesis and viral replication. TNF-alpha also functions in promoting responses to sepsis. A non-limiting example of a human TNF-alpha amino acid sequence is set forth under NCBI Reference Sequence number NP_000585.

The term “C-reactive protein” or “CRP” refers to a pentameric ring-shaped protein that is encoded by the CRP gene and is a member of the pentraxin family of proteins. CRP is synthesized by the liver and the levels of the protein increase in response to IL-6 secretion by macrophages and T cells. CRP binds to phosphocholine that is present on the surface of dead or dying cells, as well as some bacteria, thus activating the complement system and promoting phagocytosis by macrophages. Non-limiting examples of human CRP amino acid sequences are set forth under NCBI Reference Sequence numbers NP_000558, NP_001315986, and NP_001315987.

The term “interferon-gamma” or “IFN-γ” refers to a cytokine that is a member of the type II class of interferons and is encoded by the IFNG gene. IFN-γ plays important roles in innate and adaptive immunity against viral, bacterial, and protozoal infections. In particular, IFN-γ is a macrophage activator and induces expression of class II MHC molecules. IFN-γ is produced by natural killer cells, natural killer T cells, CD4⁺ Th1 cells, CD8⁺ cytotoxic T lymphocyte cells, and non-cytotoxic innate lymphoid cells. A non-limiting example of a human IFN-γ amino acid sequence is set forth under NCBI Reference Sequence number NP_000610.

The term “immunomodulatory mutation” refers to any mutation that increases or decreases the magnitude, character, and/or effectiveness of an immune response in a host cell or organism (e.g., a subject in whom an immune response against an antigen is being induced). The term includes mutations that increase the expression and/or activity of proteins involved in modulating the immune response in a host cell or organism. As a non-limiting example, an immunomodulatory mutation can increase or decrease the expression and/or activity of a cytokine (e.g., an interleukin, chemokine, interferon, lymphokine, and/or tumor necrosis factor). In some instances, an immunomodulatory mutation decreases or abolishes the function of a protein that inhibits immune function (e.g., IL-10). In other instances, an immunomodulatory mutation increases the expression or activity of an immunostimulatory protein (e.g., IL-12 or IL-15). As another non-limiting example, an immunomodulatory mutation can decrease or increase the function of a protein that is associated with antigen presentation or immune surveillance. In some instances, an immunomodulatory mutation decreases virus-mediated inhibition of major histocompatibility complex (MHC)-associated antigen presentation. As a further non-limiting example, an immunomodulatory mutation can increase or decrease the expression and/or activity of a protein that is involved in modulating the unfolded protein response (UPR). The term includes insertions, deletions, and/or substitutions of one or more nucleotides, including insertions of one or more partial or entire gene sequences, as well as deletions of partial or entire gene sequences.

The term “antigen” refers to a molecule that is capable of inducing an immune response (e.g., in a subject). While in many instances an immune response involves the production of an antibody that targets or specifically binds to the antigen, as used herein an antigen also refers to molecules that induce immune responses other than those that specifically involve the production of an antibody that targets the antigen, e.g., a cell-mediated immune response involving expansion of T cells that target antigen-derived peptides presented on the surface of target cells. The antigen can originate from a foreign organism, such as a virus or microbe (e.g., bacterial organism), or can originate from a foreign tissue. Alternatively, the antigen can originate from within a subject (i.e., a subject in which the antigen induces an immune response). As anon-limiting example, an antigen can originate from a cell in a subject that has been injured, has been infected with a pathogen (e.g. a virus or microbe such as a bacterial organism), or is aberrant or damaged (e.g., a cancer cell). The term also refers to molecules that do not necessarily induce immune responses by themselves.

The term “immune response” refers to any response that is induced (e.g., in a subject) by an antigen, including the induction of immunity against pathogens (e.g., viruses and microbes such as bacteria). Immune responses induced by recombinant polynucleotides, compositions, and methods of the present invention are typically desired, intended, and/or protective immune responses. The term includes the production of antibodies against an antigen, as well as the development, maturation, differentiation, and activation of immune cells (e.g., B cells and T cells). In some instances, an immune response comprises increasing the number or activation of MHC-E-restricted CD4⁺ and/or CD8⁺ T cells (e.g., in a subject). The term also includes increasing or decreasing the expression or activity of cytokines that are involved in regulating immune function. As another non-limiting example, an immune response can comprise increasing the expression or activity of interferon-gamma and/or tumor necrosis factor-alpha (e.g., in a subject).

Further examples of desired, intended, and/or protective immune responses that can be induced according to recombinant polynucleotides, compositions, and methods of the present invention include, but are not limited to, those involving class Ia-, class Ib-, or class II-restricted CD4⁺ T cells; class Ia-, class Ib-, or class II-restricted CD8⁺ T cells; cytokine-producing T cells (e.g., T cells that produce IFN-gamma, TNF-alpha, IL-1-beta, IL-2, IL-4, IL-IL-10, IL-13, IL-17, IL-18, or IL-23); CCR7⁻CD8⁺ T cells (e.g., effector-memory cells); CXCR5⁺ T cells (i.e., those homing to B cell follicles); CD4⁺ regulatory T cells; CD8⁺ regulatory T cells; antigen-specific T follicular helper cells; antibody production; NK cells; NKG2C⁺ NK cells; CD57⁺ NK cells; FcR-gamma-negative NK cells; and NK-CTL cells, i.e., CD8⁺ T cells expressing molecules typical of NK cells, such as NKG2A.

The term “antigen-presenting cell” or “APC” refers to a cell that displays or presents an antigen, or a portion thereof, on the surface of the cell. Typically, antigens are displayed or presented with a major histocompatibility complex (MHC) molecule. Almost all cell types can serve as APCs, and APCs are found in a large number of different tissue types. Professional APCs, such as dendritic cells, macrophages, and B cells, present antigens to T cells in a context that most efficiently leads to the T cells' activation and subsequent proliferation. Many cell types present antigens to cytotoxic T cells.

The term “infectious disease” refers to any disease or disorder caused by an organism, (e.g., viruses, bacteria, fungi, protozoa, helminths, and parasitic organisms). The term includes diseases and disorders that are transmitted from one subject to another (e.g., human to human, non-human animal to human, and human to non-human animal), as well as those caused by ingesting contaminated food or water or by exposure to pathogenic organisms (e.g., in the environment).

An “infectious disease antigen” refers to any molecule originating from an infectious disease-causing organism that can induce an immune response (e.g., in a subject). For example, an infectious disease antigen can originate from a virus, bacterium, fungus, protozoan, helminth, or parasite, and can be, for example, a bacterial wall protein, a viral capsid or structural protein (e.g., a retroviral group-specific antigen (gag) protein, such as an HIV or SIV gag protein), or a portion thereof.

The term “cancer” refers to any of various malignant neoplasms characterized by the proliferation of anaplastic cells that tend to invade surrounding tissue and metastasize to new body sites. Non-limiting examples of different types of cancer suitable for treatment using the methods and compositions of the present invention include colorectal cancer, colon cancer, anal cancer, liver cancer, ovarian cancer, breast cancer, lung cancer, bladder cancer, thyroid cancer, pleural cancer, pancreatic cancer, cervical cancer, prostate cancer, testicular cancer, bile duct cancer, gastrointestinal carcinoid tumors, esophageal cancer, gall bladder cancer, rectal cancer, appendix cancer, small intestine cancer, stomach (gastric) cancer, renal cancer (e.g., renal cell carcinoma), cancer of the central nervous system, skin cancer, oral squamous cell carcinoma, choriocarcinomas, head and neck cancers, bone cancer, osteogenic sarcomas, fibrosarcoma, neuroblastoma, glioma, melanoma, leukemia (e.g., acute lymphocytic leukemia, chronic lymphocytic leukemia, acute myelogenous leukemia, chronic myelogenous leukemia, or hairy cell leukemia), lymphoma (e.g., non-Hodgkin's lymphoma, Hodgkin's lymphoma, B-cell lymphoma, or Burkitt's lymphoma), and multiple myeloma.

The term “tumor-associated antigen” or “TAA” refers to any antigen that is produced by a tumor cell (i.e., any protein or molecule produced by a tumor cell that can induce an immune response, e.g., in a subject). TAAs include, but are not limited to, products of mutated oncogenes and mutated tumor suppressor genes, overexpressed or aberrantly expressed cellular proteins, antigens that are produced by oncogenic viruses, oncofetal antigens, altered cell surface glycolipids and glycoproteins, and antigens that are cell type-specific.

Non-limiting examples of TAAs include the melanoma-associated antigens (MAGEs). MAGE proteins contain a conserved domain that is about 200 amino acids in length and is usually located near the C-terminal end of the protein, although the conserved domain is located closer to the central portion of some MAGE proteins. Human MAGE proteins include MAGEA1, MAGEA2, MAGEA2B, MAGEA3, MAGEA4, MAGEA5, MAGEA6, MAGEA7P, MAGEA8, MAGEA9, MAGEA9B, MAGEA10, MAGEA11, MAGEA12, MAGEA13P, MAGEB1, MAGEB2, MAGEB3, MAGEB4, MAGEB5, MAGEB6, MAGEB10, MAGEB16, MAGEB17, MAGEB18, MAGEC1, MAGEC2, MAGEC3, MAGED1, MAGED2, MAGED3 (also known as “trophin” or “TRO”), MAGED4, MAGED4B, MAGEE1, MAGEE2, MAGEF1, MAGEEG1 (also known as “NSMCE3”), MAGEH1, MAGEL2, and NDN.

The protein “melanoma-associated antigen 4” or “MAGEA4” is encoded by the MAGEA4 gene in humans, located at chromosomal location Xq28. Non-limiting examples of human MAGEA4 amino acid sequences are set forth under NCBI Reference Sequence numbers NP_001011548, NP_001011549, NP_001011550, and NP_002353.

The protein “melanoma-associated antigen 10” or “MAGEA10” is encoded by the MAGEA10 gene in humans, located at chromosomal location Xq28. Non-limiting examples of human MAGEA10 amino acid sequences are set forth under NCBI Reference Sequence numbers NP_001011543, NP_001238757, and NP_066386.

The term “prostate-specific antigen” or “PSA” refers to a glycoprotein encoded by the KLK3 gene in humans, and is also known as “gamma-seminoprotein” and “kallikrein-3.” PSA is present in small quantities in the serum of men with normal prostates, but is often elevated in the presence of prostate cancer or other disorders of the prostate. Non-limiting examples of human PSA amino acid sequences are set forth under NCBI Reference Sequence numbers NP_001025218, NP_001025219, NP_001639.

The term “NY-ESO-1” refers to the cancer/testis family tumor antigen that is also known as “cancer/testis antigen 1” and is encoded by the CTAG1B gene in humans. NY-ESO-1 is highly expressed in many poor-prognosis melanomas. A non-limiting example of a human NY-ESO-1 amino acid sequence is set forth under NCBI Reference Sequence number NP_001318.1.

The term “major histocompatibility complex” or “MHC” refers to a group of cell surface proteins that are essential for recognition of foreign molecules by the adaptive immune system. The primary function of MHC molecules is to bind to antigens or antigen-derived peptides that are derived from pathogens and subsequently display the antigens on the surfaces of cells in order to facilitate recognition by T cells. MHC molecules also participate in interactions between leukocytes and other leukocytes, as well as between leukocytes and other cell types within the body. In humans, the MHC is also known as the “human leukocyte antigen complex” or “HLA complex.”

Class I MHC molecules, which predominantly present peptides from inside the cell, are encoded by the HLA-A, HLA-B, HLA-C, HLA-E, HLA-F, and HLA-G genes. HLA-A, HLA-B, and HLA-C genes are more polymorphic, while HLA-E, HLA-F, and HLA-G genes are less polymorphic. HLA-K and HLA-L are also known to exist as pseudogenes. In addition, beta-2-microglobulin is an MHC class I protein, encoded by the B2M gene. Non-limiting examples of HLA-A nucleotide sequences are set forth under NCBI Reference Sequence numbers NM_001242758 and NM_002116. A non-limiting example of an HLA-B nucleotide sequence is set forth under NCBI Reference Sequence number NM_005514. Non-limiting examples of HLA-C nucleotide sequences are set forth under NCBI Reference Sequence numbers NM_001243042 and NM_002117. A non-limiting example of an HLA-E nucleotide sequence is set forth under NCBI Reference Sequence number NM_005516. A non-limiting example of an HLA-F nucleotide sequence is set forth under NCBI Reference Sequence number NM_018950. A non-limiting example of an HLA-G nucleotide sequence is set forth under NCBI Reference Sequence number NM_002127. A non-limiting example of a B2M nucleotide sequence is set forth under NCBI Reference Sequence number NM_004048.

Class II MHC molecules, which predominantly present antigens from the outside of the cell to T lymphocytes, are encoded by the HLA-DP, HLA-D HLA-DO, HLA-DQ, and HLA-DR genes. HLA-DM genes include HLA-DMA and HLA-DMB. HLA-DO genes include HLA-DOA and HLA-DOB. HLA-DP genes include HLA-DPA1 and HLA-DPB1. HLA-DQ genes include HLA-DQA1, HLA-DQA2, HLA-DQB1, and HLA-DQB2. HLA-DR genes include HLA-DRA, HLA-DRB1, HLA-DRB3, HLA-DRB4, and HLA-DRB5. Non-limiting examples of HLA-DMA and HLA-DMB nucleotide sequences are set forth under NCBI Reference Sequence numbers NM_006120 and NM_002118, respectively. Non-limiting examples of HLA-DRA, HLA-DRB1, HLA-DRB3, HLA-DRB4, and HLA-DRB5 nucleotide sequences are set forth in NCBI Reference Sequence numbers NM_01911, NM_002124, NM_022555, NM_021983, NM_002125, respectively.

As used herein, the term “tropism” refers to the ability of a composition of the present invention (e.g., a recombinant polynucleotide of the present invention or a viral particle comprising or encoded by a recombinant polynucleotide of the present invention) to enter, infect, or replicate in a particular cell or tissue type (e.g., a target cell or tissue type found in a subject in whom an immune response against an antigen is being induced). As non-limiting examples, tropism can be broad (i.e., a recombinant polynucleotide of the present invention can enter a large number of different cell or tissue types, or a virus comprising or encoded by a recombinant polynucleotide of the present invention can infect or replicate in a large number of different cell or tissue types) or can be narrow (i.e., a recombinant polynucleotide of the present invention can enter only a small number of different cell or tissue types, or a virus comprising or encoded by a recombinant polynucleotide of the present invention can infect or replicate in only a small number of different cell or tissue types). Furthermore, as described further herein, recombinant polynucleotides of the present invention can be modified such that they possess tropism for specific desired cell or tissue type(s) (i.e., a recombinant polynucleotide can enter specific desired cell or tissue type(s), or a viral particle comprising or encoded by a recombinant polynucleotide of the present invention can enter specific desired cell or tissue type(s)). In some instances, tropism for a specific cell or tissue type is increased or imparted by the addition of a nucleic acid sequence that encodes a cellular targeting ligand.

The term “cellular targeting ligand” refers to any protein, molecule, or portion thereof that increases the ability of a composition of the present invention to enter, infect, or replicate in a specific cell or tissue type. As a non-limiting example, a cellular targeting ligand can increase the ability of a composition of the present invention (e.g., a recombinant polynucleotide of the present invention, or a viral particle comprising or encoded by a recombinant polynucleotide of the present invention) to be recognized by a specific target cell or tissue type, or to recognize a specific target cell or tissue type. Cellular targeting ligands include, but are not limited to, antibody fragments that recognize a target cell antigen, ligands that are recognized by a target cell cognate receptor, and viral capsid proteins that recognize a target cell.

As used herein, the terms “polynucleotide,” “nucleic acid,” and “nucleotide,” refer to deoxyribonucleic acids (DNA) or ribonucleic acids (RNA) and polymers thereof. The term includes, but is not limited to, single-, double-, or multi-stranded DNA or RNA, genomic DNA, cDNA, and DNA-RNA hybrids, as well as other polymers comprising purine and/or pyrimidine bases or other natural, chemically modified, biochemically modified, non-natural, synthetic, or derivatized nucleotide bases. Unless specifically limited, the term encompasses nucleic acids containing known analogs of natural nucleotides that have similar binding properties as the reference nucleic acid. Unless otherwise indicated, a particular nucleic acid sequence also implicitly encompasses conservatively modified variants thereof (e.g., degenerate codon substitutions), homologs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The term “nucleotide sequence encoding a peptide” refers to a segment of DNA, which in some embodiments may be a gene or a portion thereof, involved in producing a peptide chain. A gene will generally include regions preceding and following the coding region (leader and trailer) involved in the transcription/translation of the gene product and the regulation of the transcription/translation. A gene can also include intervening sequences (introns) between individual coding segments (exons). Leaders, trailers, and introns can include regulatory elements that are necessary during the transcription and the translation of a gene (e.g., promoters, terminators, translational regulatory sequences such as ribosome binding sites and internal ribosome entry sites, enhancers, silencers, insulators, boundary elements, replication origins, matrix attachment sites and locus control regions, etc.). A “gene product” can refer to either the mRNA or protein expressed from a particular gene.

The terms “expression” and “expressed” in the context of a gene refer to the transcriptional and/or translational product of the gene. The level of expression of a DNA molecule in a cell may be assessed on the basis of either the amount of corresponding mRNA that is present within the cell or the amount of protein encoded by that DNA produced by the cell.

The term “recombinant” when used with reference, e.g., to a polynucleotide, protein, vector, or cell, indicates that the polynucleotide, protein, vector, or cell has been modified by the introduction of a heterologous nucleic acid or protein or the alteration of a native nucleic acid or protein, or that the cell is derived from a cell so modified. For example, recombinant polynucleotides contain nucleic acid sequences that are not found within the native (non-recombinant) form of the polynucleotide.

The terms “vector” and “expression vector” refer to a polynucleotide construct, generated recombinantly or synthetically, with a series of specified nucleic acid elements that permit transcription of a particular nucleic acid sequence (e.g., within a polynucleotide comprising a CMV genome or a portion thereof (e.g., a CMV genome or a portion thereof comprising one or more immunomodulatory mutations) and an antigen) in a host cell. As used herein, the term “CMV vector” or “CMV-based vector” refers to a vector that is derived from or comprises a polynucleotide (e.g., recombinant polynucleotide) comprising a CMV genome or a portion thereof. Typically, a vector includes a nucleic acid sequence to be transcribed, operably linked to a promoter. Other elements that may be present in a vector include those that enhance transcription (e.g., enhancers) and terminate transcription (e.g., terminators), those that confer certain binding affinity or antigenicity to a protein (e.g., recombinant protein) produced from the vector, and those that enable replication of the vector and its packaging into a viral particle (e.g., a CMV particle). Recombinant polynucleotides of the present invention that are CMV-based vectors can be used as viral vaccine vectors.

The terms “polypeptide,” “peptide,” and “protein” are used interchangeably herein to refer to a polymer of amino acid residues. All three terms apply to amino acid polymers in which one or more amino acid residues are an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins, wherein the amino acid residues are linked by covalent peptide bonds.

The terms “subject,” “individual,” and “patient” are used interchangeably herein to refer to a vertebrate, preferably a mammal, more preferably a human. Mammals include, but are not limited to, murines, mice, rats, simians, humans, farm animals, sport animals, and pets. Tissues, cells and their progeny of a biological entity obtained in vivo or cultured in vitro are also encompassed.

As used herein, the term “administering” includes oral administration, topical contact, administration as a suppository, intravenous, intraperitoneal, intramuscular, intralesional, intratumoral, intrathecal, intranasal, intraosseous, or subcutaneous administration to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arterial, intradermal, subcutaneous, intraperitoneal, intraventricular, intraosseous, and intracranial. Other modes of delivery include, but are not limited to, the use of liposomal formulations, intravenous infusion, transdermal patches, etc.

The term “treating” refers to an approach for obtaining beneficial or desired results including, but not limited to, a therapeutic benefit and/or a prophylactic benefit. “Therapeutic benefit” means any therapeutically relevant improvement in or effect on one or more diseases, conditions, or symptoms under treatment. Therapeutic benefit can also mean to effect a cure of one or more diseases, conditions, or symptoms under treatment. Furthermore, therapeutic benefit can also mean to increase survival. For prophylactic benefit, the compositions may be administered to a subject at risk of developing a particular disease, condition, or symptom, or to a subject reporting one or more of the physiological symptoms of a disease, even though the disease, condition, or symptom may not yet be present.

The term “survival” refers to a length of time following the diagnosis of a disease and/or beginning or completing a particular course of therapy for a disease (e.g., cancer or an infectious disease). The term “overall survival” includes the clinical endpoint describing patients who are alive for a defined period of time after being diagnosed with or treated for a disease, such as cancer. The term “disease-free survival” includes the length of time after treatment for a specific disease during which a patient survives with no sign of the disease (e.g., without known recurrence). In certain embodiments, disease-free survival is a clinical parameter used to evaluate the efficacy of a particular therapy, which in some instances is measured in units of 1 or 5 years. The term “progression-free survival” includes the length of time during and after treatment for a specific disease in which a patient is living with the disease without additional symptoms of the disease. In some embodiments, survival is expressed as a median or mean value.

The term “therapeutically effective amount” or “sufficient amount” refers to the amount of a recombinant polynucleotide or composition that is sufficient to effect beneficial or desired results. The therapeutically effective amount may vary depending upon one or more of: the subject and disease condition being treated, the weight and age of the subject, the severity of the disease condition, the immune status of the subject, the manner of administration and the like, which can readily be determined by one of ordinary skill in the art. The specific amount may vary depending on one or more of: the particular agent chosen, the target cell type, the location of the target cell in the subject, the dosing regimen to be followed, whether it is administered in combination with other compounds, timing of administration, and the physical delivery system in which it is carried.

For the purposes herein an effective amount is determined by such considerations as may be known in the art. The amount must be effective in achieving the desired therapeutic effect in a subject suffering from a disease such as an infectious disease or cancer. The desired therapeutic effect may include, for example, amelioration of undesired symptoms associated with the disease, prevention of the manifestation of such symptoms before they occur, slowing down the progression of symptoms associated with the disease, slowing down or limiting any irreversible damage caused by the disease, lessening the severity of or curing the disease, or improving the survival rate or providing more rapid recovery from the disease. Further, in the context of prophylactic treatment the amount may also be effective to prevent the development of the disease.

The term “pharmaceutically acceptable carrier” refers to a substance that aids the administration of an active agent to a cell, an organism, or a subject. “Pharmaceutically acceptable carrier” refers to a carrier or excipient that can be included in the compositions of the invention and that causes no significant adverse toxicological effect on the patient. Non-limiting examples of pharmaceutically acceptable carriers include water, sodium chloride (NaCl), normal saline solutions, lactated Ringer's, normal sucrose, normal glucose, binders, fillers, disintegrants, lubricants, coatings, sweeteners, flavors and colors, liposomes, dispersion media, microcapsules, cationic lipid carriers, isotonic and absorption delaying agents, and the like. The carrier may also comprise or consist of substances for providing the formulation with stability, sterility and isotonicity (e.g. antimicrobial preservatives, antioxidants, chelating agents and buffers), for preventing the action of microorganisms (e.g. antimicrobial and antifungal agents, such as parabens, chlorobutanol, phenol, sorbic acid and the like) or for providing the formulation with an edible flavor, etc. In some instances, the carrier is an agent that facilitates the delivery of a polypeptide, fusion protein, or polynucleotide to a target cell or tissue. One of skill in the art will recognize that other pharmaceutical carriers are useful in the present invention.

The term “vaccine” refers to a biological composition that, when administered to a subject, has the ability to produce an acquired immunity to a particular pathogen or disease in the subject. Typically, one or more antigens, fragments of antigens, or polynucleotides encoding antigens or fragments of antigens that are associated with the pathogen or disease of interest are administered to the subject. Vaccines can comprise, for example, inactivated or attenuated organisms (e.g., bacteria or viruses), cells, proteins that are expressed from or on cells (e.g., cell surface or other proteins produced by cells (e.g., tumor cells)), proteins that are produced by organisms (e.g., toxins), or portions of organisms (e.g., viral envelope proteins or viral genes encoding various antigens). In some instances, cells are engineered to express proteins such that, when administered as a vaccine, they enhance the ability of a subject to acquire immunity to that particular cell type (e.g., enhance the ability of a subject to acquire immunity to a cancer cell or to an organism that causes an infectious disease such as a virus, a bacterium, a fungal organism, a protozoan, or a helminth). As used herein, the term “vaccine” includes, but is not limited to, recombinant polynucleotides of the present invention (e.g., CMV-based vectors that can be used in viral vector vaccines), as well as viral particles, host cells, and pharmaceutical compositions that comprise recombinant polynucleotides of the present invention.

The term “unfolded protein response” or “UPR” refers to a cellular stress response that is conserved across many species, including mammals, yeast, and worms, and is activated in response to the accumulation of unfolded or misfolded proteins in the endoplasmic reticulum of a cell. Initially, the UPR functions to decrease protein translation, degrade misfolded proteins, and facilitate activation of signaling pathways that lead to increased production of molecular chaperones. If the UPR is sustained, eventually its functioning can induce apoptosis. Within the lumen of the endoplasmic reticulum, the UPR is initiated as BIP/Grp78 chaperones, which normally associate with the luminal domains of UPR-activating transmembrane proteins (thus preventing activation of the UPR), become dissociated from these proteins as BIP/Grp78 is forced to associate with unfolded or misfolded proteins. Cytomegaloviruses contain genes that inhibit the UPR (e.g., Human cytomegalovirus UL50, Rhesus cytomegalovirus Rh81, and Mouse cytomegalovirus M50), the protein products of which suppress IRE1-mediated XBP1 splicing via conserved sequences located at their N-terminal ends.

The term “group-specific antigen” or “gag” refers to a protein encoded by a retroviral gag gene. Gag genes encode the core structural proteins of retroviruses. In human immunodeficiency virus (HIV) and the closely related simian immunodeficiency virus (SIV), the gag gene encodes a gag polyprotein precursor (known in the case of HIV as Pr55^{Gag}), which is subsequently proteolytically processed into the p17 matrix protein (MA), the p24 capsid protein (CA), the p7 nucleocapsid protein (NC), the SP1 and SP2 spacer peptides, and the p6 polypeptide that is located at the N-terminus of the gag polyprotein. Non-limiting examples of HIV and SIV gag protein sequences are set forth under UniProt reference numbers P04591 and P89153, respectively.

### III. Recombinant Polynucleotides

In one aspect, recombinant polynucleotides are provided. The recombinant polynucleotides find utility for, among other things, use as viral vectors and viral vector vaccines. Thus, in some aspects, viral vectors comprising a recombinant polynucleotide are provided herein. In other aspects, viral vaccines comprising a recombinant polynucleotide are provided herein. In some embodiments, the recombinant polynucleotide comprises a cytomegalovirus (CMV) genome, or a portion thereof, and a nucleic acid sequence encoding an antigen. In some embodiments, the CMV genome or portion thereof comprises one or more immunomodulatory mutations. In some embodiments, the one or more immunomodulatory mutations comprise a mutation within a nucleic acid sequence encoding a protein that has interleukin-10 (IL-10)-like activity. The one or more immunomodulatory mutations can be located, for example, in a protein coding region of the nucleic acid sequence encoding the protein that has IL-10-like activity, in a regulatory region that controls expression of the protein that has IL-10-like activity, or both. In some embodiments, the nucleic acid sequence encoding the antigen is located within the CMV genome or portion thereof. In other embodiments, the nucleic acid sequence encoding the antigen is located outside of the CMV genome or portion thereof (e.g., 5′ and/or 3′ of the CMV genome or portion thereof). In some embodiments, nucleic acid sequences encoding antigen(s) are located both inside and outside (e.g., 5′ to and/or 3′ to) of the CMV genome or portion thereof. In some embodiments, the recombinant polynucleotide comprises 1, 2, 3, 4, 5, or more CMV genomes (or portions thereof). When the recombinant polynucleotide comprises more than one CMV genome or portion thereof, immunomodulatory mutations in nucleic acid sequences encoding proteins that have IL-10-like activity can be made in one, some, or all of the CMV genomes or portions thereof (e.g., one, some, or all of the nucleic acid sequences encoding proteins that have IL-10-like activity).

In some embodiments, the CMV is a CMV that can infect human cells. In particular embodiments, the CMV is a CMV that can replicate in human cells. In some instances, the CMV is a CMV that can only enter or replicate in human cells. In some embodiments, the CMV is a CMV that can infect non-human primate cells (e.g., simian cells, chimpanzee cells, or rhesus macaque cells). In particular embodiments, the CMV is a CMV that can replicate in non-human primate cells. In some instances, the CMV is a CMV that can only enter or replicate in non-human primate cells. In some embodiments, the CMV is a CMV that can infect rodent cells (e.g., mouse cells or rat cells). In particular embodiments, the CMV is a CMV that can replicate in rodent cells. In some instances, the CMV is a CMV that can only enter or replicate in rodent cells. In some embodiments, the CMV is selected from the group consisting of Human cytomegalovirus (HCMV), Simian cytomegalovirus (SCCMV or AGMCMV), Baboon cytomegalovirus (BaCMV), Owl monkey cytomegalovirus (OMCMV), Squirrel monkey cytomegalovirus (SMCMV), and Rhesus cytomegalovirus (RhCMV). Non-limiting examples of suitable viral genomes include those set forth under NCBI Reference Sequence numbers NC_006273.2 (HCMV), FJ483969.2 (SCCMV), NC_006150.1 (RhCMV), AY186194.1 (RhCMV strain 68-1), and DQ120516.1 (Cercopithecine herpesvirus 8 isolate CMV 180.92).

In some embodiments, the protein that has IL-10-like activity is human CMV IL-10 (HCMVIL-10) or rhesus macaque CMV IL-10 (RhCMVIL-10). Immunomodulatory mutations can be introduced into genes for other (e.g., homologous) proteins, such as the genes that encode proteins having IL-10-like activity in SCCMV/AGMCMV, BaCMV, OMCMV, or SMCMV, depending on the particular CMV genome or portion thereof being used to construct a recombinant polynucleotide of the present invention. In some embodiments, the protein that has IL-10-like activity is encoded by the nucleic acid sequence set forth under SEQ ID NO:11 and/or 12.

Mutations introduced into recombinant polynucleotides of the present invention as described herein, including immunomodulatory mutations, can comprise deletions, insertions, and/or substitutions (e.g., conservative or non-conservative substitutions) of one or more nucleotides. In some embodiments, a mutation (e.g., an immunomodulatory mutation) comprises the insertion of a gene, or a portion of a gene. In other embodiments, a mutation comprises an insertion of a nucleic acid sequence that encodes a protein, or a portion of a protein. In some embodiments, a mutation comprises a deletion of an entire gene sequence, or a portion thereof. As a non-limiting example, one, two or more exons of a gene can be deleted. In some embodiments, a recombinant polynucleotide of the present invention comprises the deletion of the first two exons of a nucleic acid sequence encoding a protein that has IL-10-like activity (e.g., the first two exons of RhCMVIL-10 are deleted).

Mutations introduced into recombinant polynucleotides of the present invention as described herein, including immunomodulatory mutations, can increase or decrease the expression (e.g., mRNA and/or protein expression) and/or activity of a gene. In some embodiments, a mutation within a nucleic acid sequence encoding a protein that has IL-10-like activity (e.g., a mutation comprising the deletion of the first two exons of a nucleic acid sequence encoding a protein that has IL-10-like activity) reduces or inactivates the activity of the protein having IL-10-like activity. In some embodiments, the reduction or inactivation of the protein having IL-10-like activity produces a synergistic effect when combined with one or more other immunomodulatory mutations.

The antigen encoded by a recombinant polynucleotide of the present invention can be any antigen, so long as it produces an immune response against the desired cell type or pathogenic organism. In some embodiments, the antigen is a non-CMV antigen. In some embodiments, the antigen is an infectious disease antigen. In other embodiments, the antigen is a tumor-associated antigen (TAA).

In some embodiments, the infectious disease antigen is a bacterial infectious disease antigen. In some embodiments, the infectious disease antigen is a viral infectious disease antigen. In some embodiments, the infectious disease antigen is a fungal infectious disease antigen. In some embodiments, the infectious disease antigen is a protozoal infectious disease antigen. In some embodiments, the infectious disease antigen is a helminthic infectious disease antigen. In some embodiments, the infectious disease antigen is a bacterial, viral, fungal, protozoal, and/or helminthic infectious disease antigen. In some cases, the antigen is from a parasite. Non-limiting examples of suitable viral infectious disease antigens are those derived from simian immunodeficiency virus (SIV), human immunodeficiency virus (HIV), hepatitis C virus, herpes simplex virus, Epstein-Barr virus, or any combination thereof. As further non-limiting examples, the infectious disease antigen can comprise a retroviral group-specific antigen (gag) protein (e.g., an HIV or SIV gag protein). As yet another non-limiting example, the infectious disease antigen can be a bacterial infectious disease antigen from Mycobacterium tuberculosis.

A tumor-associated antigen (TAA) can be derived from any cancer cell. TAAs include, but are not limited to, products of mutated oncogenes and mutated tumor suppressor genes, overexpressed or aberrantly expressed cellular proteins, antigens that are produced by oncogenic viruses, oncofetal antigens, altered cell surface glycolipids and glycoproteins, antigens that are aberrantly processed in tumor cells for presentation on MHC molecules, and antigens that are tumor cell type-specific. In some embodiments, a TAA is one that newly arises in a tumor (e.g., a subject's tumor). Such neoantigens can arise, for example, as a consequence of a tumor-specific mutation. In some embodiments, a TAA is a cell surface protein (e.g., that is normally present on the surface of a cell), or a portion thereof, that is altered as a consequence of a mutation in a gene encoding the cell surface protein.

A TAA can be derived from, for example, a colorectal cancer cell, a colon cancer cell, an anal cancer cell, a liver cancer cell, an ovarian cancer cell, a breast cancer cell, a lung cancer cell, a bladder cancer cell, a thyroid cancer cell, a pleural cancer cell, a pancreatic cancer cell, a cervical cancer cell, a prostate cancer cell, a testicular cancer cell, a bile duct cancer cell, a gastrointestinal carcinoid tumor cell, an esophageal cancer cell, a gall bladder cancer cell, a rectal cancer cell, an appendix cancer cell, a small intestine cancer cell, a stomach (gastric) cancer cell, a renal cancer (e.g., renal cell carcinoma) cell, a central nervous system cancer cell, a skin cancer cell, an oral squamous cell carcinoma cell, a choriocarcinoma cell, a head and neck cancer cell, a bone cancer cell, an osteogenic sarcoma cell, a fibrosarcoma cell, a neuroblastoma cell, a glioma cell, a melanoma cell, a leukemia (e.g., acute lymphocytic leukemia, chronic lymphocytic leukemia, acute myelogenous leukemia, chronic myelogenous leukemia, or hairy cell leukemia) cell, a lymphoma (e.g., non-Hodgkin's lymphoma, Hodgkin's lymphoma, B-cell lymphoma, or Burkitt's lymphoma) cell, a multiple myeloma cell, or any combination thereof. In particular embodiments, the TAA is derived from an ovarian cancer cell, a melanoma cell, a prostate cancer cell, or a combination thereof.

Non-limiting examples of TAAs that can be encoded by nucleic acid sequences in recombinant polynucleotides of the present invention include the melanoma-associated antigens (MAGEs). MAGE proteins contain a conserved domain that is about 200 amino acids in length and is usually located near the C-terminal end of the protein, although the conserved domain is located closer to the central portion of some MAGE proteins. Human MAGE proteins include MAGEA1, MAGEA2, MAGEA2B, MAGEA3, MAGEA4, MAGEA5, MAGEA6, MAGEA7P, MAGEA8, MAGEA9, MAGEA9B, MAGEA10, MAGEA11, MAGEA12, MAGEA13P, MAGEB1, MAGEB2, MAGEB3, MAGEB4, MAGEB5, MAGEB6, MAGEB10, MAGEB16, MAGEB17, MAGEB18, MAGEC1, MAGEC2, MAGEC3, MAGED1, MAGED2, MAGED3 (also known as “trophin” or “TRO”), MAGED4, MAGED4B, MAGEE1, MAGEE2, MAGEF1, MAGEEG1 (also known as “NSMCE3”), MAGEH1, MAGEL2, and NDN. Additional non-limiting examples of TAAs that are useful for the present invention include NY-ESO-1 and prostate-specific antigen (PSA). In some embodiments, the TAA is a neoantigen.

In some embodiments, a recombinant polynucleotide of the present invention comprises nucleic acid sequences(s) encoding antigen(s) selected from the group consisting of MAGEA4, MAGEA10, NY-ESO-1, PSA, and a combination thereof.

In order to improve the magnitude and/or character of an immune response induced (e.g., in a subject) by a recombinant polynucleotide or other composition of the present invention, the one or more immunomodulatory mutations can further comprise a mutation that increases the expression or activity of an immunostimulatory protein. In some embodiments, the one or more immunomodulatory mutations further comprise a nucleic acid sequence that encodes an immunostimulatory protein (e.g., the insertion of nucleic acid sequence encoding an immunostimulatory protein). As used herein, the term “immunostimulatory protein” refers to any protein that increases the magnitude of an immune response (e.g., in a subject) and/or changes the character of an immune response such that acquired immunity (e.g., against a desired cell type or pathogen) is enhanced.

As a non-limiting example, the immunostimulatory protein can be a cytokine. In some embodiments, the cytokine is an interleukin. In some embodiments, the cytokine is a chemokine. In some embodiments, the cytokine is an interferon (e.g., a type I interferon, type II interferon (interferon-gamma in humans), and/or another type II interferon). In some embodiments, the cytokine is a lymphokine. In some embodiments, the cytokine is a tumor necrosis factor (e.g., tumor necrosis factor-alpha). In some embodiments, the cytokine is an interleukin, a chemokine, an interferon, a lymphokine, a tumor necrosis factor, or any combination thereof. In particular embodiments, the cytokine encoded by a nucleic acid sequence within a recombinant polynucleotide of the present invention comprises an interleukin. Suitable interleukins include those that stimulate the immune response such as interleukin-2 (IL-2), interleukin-12 (IL-12), interleukin-15 (IL-15), and/or a combination thereof.

Additional immunomodulatory mutations that can be introduced into recombinant polynucleotides of the present invention include mutations introduced into the Rh182, Rh183, Rh184, Rh185, Rh186, Rh187, Rh188, and/or Rh189 regions of the RhCMV genome, US2, US3, US4, US5, US6, US7, US8, US9, US10, and/or US11 of the HCMV genome, and homologs thereof (see, e.g., Hansen et al. J. Virol. (2003) 77:6620-6636). Introducing mutations into the Rh182, Rh184, Rh185, or Rh189 regions of RhCMV or the US2, US3, US6, or US11 regions of HCMV are useful, for example, for reducing the ability of CMV to inhibit antigen presentation by major histocompatibility complex (MHC) molecules (e.g., class I and/or class II MHC molecules). Rh187 and US8 are involved in binding MHC molecules (see, e.g., Tirabassi et al. J. Virol. (2002) 76:6832-6835) and thus can be used to modulate MHC-associated antigen presentation.

In some instances, it is useful to increase tropism for particular target cell or tissue type(s). This can be achieved, for example, by introducing mutation(s) into the recombinant polynucleotide that increase tropism for the desired cell or tissue type(s). In some embodiments, a mutation that increases or imparts tropism (e.g., for a target cell or tissue) is introduced into the recombinant polynucleotide comprising the CMV genome or portion thereof. Non-limiting examples of suitable target cells are antigen-presenting cells, tumor cells, fibroblasts, epithelial cells, endothelial cells, and combinations thereof. Suitable antigen-presenting cells include, but are not limited to, dendritic cells, macrophages, and B cells. In particular embodiments, the antigen-presenting cell is a dendritic cell.

Another approach for increasing or imparting target cell or tissue tropism is to introduce mutations into nucleic acid sequences (e.g., within the recombinant polynucleotide comprising the CMV genome or portion thereof) that result in the modification of proteins, or portions thereof, that are positioned on the outside of the CMV virion. As a non-limiting example, a CMV envelope protein can be modified by the addition of a blocking domain that decreases or prevents entry of the CMV into a cell, unless the blocking domain is cleaved, e.g., by a protease expressed by a target cell. For example, proteases such as matrix metalloproteases that are expressed by tumor cells of interest can cleave off envelope protein blocking domains, thereby allowing CMV entry only into the tumor cells of interest and increasing tropism for those target cells.

Furthermore, tropism for a target cell or tissue type can be increased or imparted by introducing a nucleic acid sequence (e.g., within the recombinant polynucleotide comprising the CMV genome or portion thereof) that encodes a cellular targeting ligand. To serve as non-limiting examples, a cellular targeting ligand can be an antibody or fragment thereof that recognizes a target cell antigen, a ligand that is recognized by a target cell cognate receptor, a viral capsid protein that recognizes a target cell, or any combination thereof. Non-limiting examples of antibodies and fragments thereof that recognize target cell antigens include antibodies that recognize dendritic cell-specific intercellular adhesion molecule-3-grabbing non-integrin (DC-SIGN; also known as CD209), CD40, CD64, class II MHC molecules, and DEC205 (also known as CD205), all of which are expressed by dendritic cells.

Suitable ligands that are recognized by target cell cognate receptors include, but are not limited to, CD40L (which is also known as CD154 and binds to CD40, which is expressed, e.g., by dendritic cells) and ICAM3 (which has high affinity for DC-SIGN that is expressed by, e.g., APCs such as dendritic cells). In particular embodiments, the cellular targeting ligand is CD40L/CD154.

Non-limiting examples of viral capsid proteins that are recognized by target cells include Ad16, Ad26, Ad35, or Ad37 virus fiber proteins (i.e., for targeting dendritic cells) and Sindbis virus envelope glycoproteins (which can also be used for targeting dendritic cells, via DC-SIGN).

Additional mutations that can be introduced into a recombinant polynucleotide of the present invention (e.g., to increase target tropism) include mutations (e.g., deletions) within the Rh13.1, Rh61/Rh60, Rh157.4, Rh157.5, and/or Rh157.6 genes of RhCMV, or homologs thereof. Human CMV orthologs of Rh13.1, Rh61/Rh60, Rh157.4, Rh157.5, and Rh157.6 include, but are not limited to, RL13, UL36 (also known as viral inhibitor of caspase-8-induced apoptosis (vICA)), UL130, UL128, and UL131, respectively. Rh13.1 and RL13 are involved in, for example, inhibiting growth of the virus in fibroblasts. Rh157.4, Rh157.5, Rh157.6, UL130, UL128, and UL131 encode three components of an entry receptor for non-fibroblast cells (e.g., endothelial and epithelial cells).

CMV genomes typically contain nucleic acid sequences that encode for proteins that suppress the unfolded protein response (UPR) in a host. In some instances, it is desirable to further suppress the UPR (e.g., in a host being administered a recombinant polynucleotide or other composition of the present invention), for example, by further increasing the expression or activity of a CMV protein that suppresses the UPR. In other instances, it is desirable to decrease or eliminate the ability of CMV to suppress the UPR, for example, by decreasing the expression or activity of a CMV protein that suppresses the UPR. CMV proteins that are known to suppress the UPR include, Human cytomegalovirus UL50, Rhesus cytomegalovirus Rh81, or Mouse cytomegalovirus M50. In some embodiments, a recombinant polynucleotide of the present invention comprises or further comprises an immunomodulatory mutation that increases or decreases the UPR (e.g., in a subject). In varying embodiments, the immunomodulatory mutation that increases or decreases the UPR decreases or increases the expression and/or activity of Human cytomegalovirus UL50, Rhesus cytomegalovirus Rh81, Mouse cytomegalovirus M50, or a homolog thereof.

In some embodiments, a recombinant polynucleotide of the present invention contains a nucleic acid sequence that encodes a selectable marker. The nucleic acid sequence can be located within the CMV genome or portion thereof, outside of (e.g., 5′ and/or 3′ to) the CMV genome or portion thereof, or a combination thereof. A selectable marker is useful, for example, when a polynucleotide of the present invention is being recombinantly modified, especially when it is desirable to screen a population of modified polynucleotides (e.g., using bacterial, yeast, plant, or animal cells) for those that have incorporated the desired modification(s) (e.g., insertion, deletion, or a combination thereof). As a non-limiting example and as described in the Examples section, one or more exons of a gene of interest (e.g., a CMV gene for a protein that has IL-10-like activity) in a recombinant polynucleotide of the present invention can be deleted by recombinantly replacing the nucleic acid sequence encoding the exon(s) with a nucleic acid sequence encoding a selectable marker (e.g., an antibiotic resistance gene such as a gene that encodes resistance to Zeocin). The nucleic acid sequence encoding the selectable marker can optionally be under the control of a promoter (e.g., EM7 promoter) and/or other regulatory sequence(s). Whether the polynucleotide is recombinantly modified within a cell (e.g., a bacterial cell, for example, using Red/ET recombination) or is recombinantly modified and subsequently introduced into a cell (e.g., bacterial, yeast, plant, or animal cell) for screening, the selectable marker can be used to identify which cells contain polynucleotides that have incorporated a modification of interest. Treating the cells that contain the recombinant polynucleotides with Zeocin will identify which cells contain recombinant polynucleotides that have incorporated the antibiotic resistance gene (i.e., the cells that survive after Zeocin treatment must have incorporated the antibiotic resistance gene). If desired, the recombinant polynucleotides can be further screened (e.g., purified from the cells, amplified, and sequenced), in order to verify that the desired modification has been recombinantly introduced into the polynucleotide at the correct position.

When the selectable marker is an antibiotic resistance gene, the gene can confer resistance to Zeocin, ampicillin, tetracycline, chloramphenicol, or another appropriate antibiotic that will be known to one of skill in the art. In some embodiments, a selectable marker is used that produces a visible phenotype, such as the color of an organism or population of organisms. As a non-limiting example, the phenotype can be examined by growing the organisms (e.g., cells or other organisms that contain the recombinant polynucleotide) and/or their progeny under conditions that result in a phenotype, wherein the phenotype may not be visible under ordinary growth conditions.

In some embodiments, the selectable marker used for identifying cells that contain a polynucleotide containing a modification of interest is a fluorescently tagged protein, a chemical stain, a chemical indicator, or a combination thereof. In other embodiments, the selectable marker responds to a stimulus, a biochemical, or a change in environmental conditions. In some instances, the selectable marker responds to the concentration of a metabolic product, a protein product, a drug, a cellular phenotype of interest, a cellular product of interest, or a combination thereof.

Commonly, recombinant polynucleotides of the present invention will contain one or more regulatory sequences. The regulatory sequence(s) can be located within the CMV genome or portion thereof, outside of (e.g., 5′ and/or 3′ to) the CMV genome or portion thereof, or a combination thereof. In some embodiments, the regulatory sequence(s) are recombinantly introduced into the polynucleotide. For example, one or more regulatory sequences can be introduced into a CMV genome or portion thereof that are not present in the natural CMV genome-encoding sequence. Alternatively, a regulatory sequence that is present in the natural CMV genome-encoding sequence can be deleted or otherwise modified.

In some embodiments, the regulatory sequence(s) control the expression and/or activity of a gene or region within a CMV genome or portion thereof. In some embodiments, the regulatory sequence(s) control the expression and/or activity of an antigen-encoding sequence. In some embodiments, the regulatory sequence(s) control the expression and/or activity of an immunostimulatory protein-encoding sequence. In some embodiments, the regulatory sequence(s) control the expression and/or activity of a selectable marker-encoding sequence. In some embodiments, the regulatory sequence(s) control the expression and/or activity of a gene or region within a CMV genome or portion thereof an antigen-encoding sequence, an immunostimulatory protein-encoding sequence, a selectable marker-encoding sequence, a variant thereof, or a combination thereof.

Depending on the cell system used, the regulatory sequence(s) may comprise transcription and translation control elements, including promoters, transcription enhancers, transcription terminators, and the like. Useful promoters can be derived from viruses or any other organism, e.g., prokaryotic or eukaryotic organisms. Promoters may also be inducible (i.e., capable of responding to environmental factors and/or external stimuli that can be artificially controlled). Non-limiting examples of promoters include unmodified and modified bacterial T7 promoters such as the EM7 promoter, the EF1α promoter, RNA polymerase II promoters (e.g., pGAL7 and pTEF1), RNA polymerase III promoters (e.g., RPR-tetO, SNR52, and tRNA-tyr), the SV40 early promoter, mouse mammary tumor virus long terminal repeat (LTR) promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, a human U6 small nuclear promoter (U6), an enhanced U6 promoter, a human H1 promoter (H1), etc. Suitable polyadenylation sequences and terminators include, but are not limited to, SV40, hGH, BGH, rbGlob SNR52, and RPR polyadenylation and terminator sequences. Additionally, various primer binding sites may be incorporated into a vector to facilitate vector cloning, sequencing, genotyping, and the like. In some embodiments, a “CAG promoter” is used as the regulatory sequence, which comprises a CMV early enhancer, a chicken beta-actin gene promoter, a first exon of the chicken beta-actin gene, a first intron of the chicken beta-actin gene, and a splice acceptor of the rabbit beta-globin gene. Other suitable promoter, enhancer, terminator, and primer binding sequences will readily be known to one of skill in the art.

The size of a recombinant polynucleotide of the present invention will depend on the CMV genome(s), or portion(s) thereof, being included, the particular antigen(s) that are being encoded, additional immunomodulatory mutations such as the inclusion of immunostimulatory protein-encoding sequences, etc. In some embodiments, the recombinant polynucleotide is between about 100 kilobases and about 300 kilobases (e.g., about 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, or about 300 kilobases) in length. In some embodiments, the recombinant polynucleotide is greater than about 300 kilobases in length.

In some embodiments, the recombinant polynucleotide is about 100 kilobases to about 300 kilobases, about 100 kilobases to about 280 kilobases, about 100 kilobases to about 260 kilobases, about 100 kilobases to about 240 kilobases, about 100 kilobases to about 220 kilobases, about 100 kilobases to about 200 kilobases, about 100 kilobases to about 180 kilobases, about 100 kilobases to about 160 kilobases, about 100 kilobases to about 140 kilobases, about 100 kilobases to about 120 kilobases, about 120 kilobases to about 300 kilobases, about 120 kilobases to about 280 kilobases, about 120 kilobases to about 260 kilobases, about 120 kilobases to about 240 kilobases, about 120 kilobases to about 220 kilobases, about 120 kilobases to about 200 kilobases, about 120 kilobases to about 180 kilobases, about 120 kilobases to about 160 kilobases, about 120 kilobases to about 140 kilobases, about 140 kilobases to about 300 kilobases, about 140 kilobases to about 280 kilobases, about 140 kilobases to about 260 kilobases, about 140 kilobases to about 240 kilobases, about 140 kilobases to about 220 kilobases, about 140 kilobases to about 200 kilobases, about 140 kilobases to about 180 kilobases, about 140 kilobases to about 160 kilobases, about 160 kilobases to about 300 kilobases, about 160 kilobases to about 280 kilobases, about 160 kilobases to about 260 kilobases, about 160 kilobases to about 240 kilobases, about 160 kilobases to about 220 kilobases, about 160 kilobases to about 200 kilobases, about 160 kilobases to about 180 kilobases, about 180 kilobases to about 300 kilobases, about 180 kilobases to about 280 kilobases, about 180 kilobases to about 260 kilobases, about 180 kilobases to about 240 kilobases, about 180 kilobases to about 220 kilobases, about 180 kilobases to about 200 kilobases, about 200 kilobases to about 300 kilobases, about 200 kilobases to about 280 kilobases, about 200 kilobases to about 260 kilobases, about 200 kilobases to about 240 kilobases, about 200 kilobases to about 220 kilobases, about 220 kilobases to about 300 kilobases, about 220 kilobases to about 280 kilobases, about 220 kilobases to about 260 kilobases, about 220 kilobases to about 240 kilobases, about 240 kilobases to about 300 kilobases, about 240 kilobases to about 280 kilobases, about 240 kilobases to about 260 kilobases, about 260 kilobases to about 300 kilobases, about 260 kilobases to about 280 kilobases, or about 280 kilobases to about 300 kilobases in length.

In some embodiments, the antigen encoded by the recombinant polynucleotide of the present invention is expressed as part of a fusion protein. In particular embodiments, the fusion protein comprises the antigen and a tag. Non-limiting examples of tags include StrepTag (StrepII) (8 a.a.); SBP (38 a.a.); biotin carboxyl carrier protein or BCCP (100 a.a.); epitope tags such as FLAG (8 a.a.), 3xFLAG (22 a.a.), and myc (22 a.a.); S-tag (Novagen) (15 a.a.); Xpress (Invitrogen) (25 a.a.); eXact (Bio-Rad) (75 a.a.); HA (9 a.a.); VSV-G (11 a.a.); Protein A/G (280 a.a.); HIS (6-10 a.a.) (SEQ ID NO: 15); glutathione s-transferase or GST (218 a.a.); maltose binding protein or MBP (396 a.a.); CBP (28 a.a.); CYD (5 a.a.); HPC (12 a.a.); CBD intein-chitin binding domain (51 a.a.); Trx (Invitrogen) (109 a.a.); NorpA (5 a.a.); and NusA (495 a.a.). In some embodiments, the antigen is expressed as fusion protein that comprises the antigen and a FLAG tag. In some instances, the antigen comprises a gag-FLAG fusion protein.

In another aspect, viral particles (e.g., CMV particles) are provided. In some embodiments, the viral particle comprises a recombinant polynucleotide of the present invention, or a plurality thereof. In some embodiments, the viral particle is one that replicates in and/or is released from an infected, transfected, or transformed host cell.

In yet another aspect, host cells are provided. In some embodiments, the host cell comprises a recombinant polynucleotide of the present invention. In other embodiments, the host cell comprises a viral particle of the present invention (e.g., a viral particle comprising a recombinant polynucleotide of the present invention). In some embodiments, the host cell comprises a recombinant polynucleotide of the present invention and/or a viral particle of the present invention. In some embodiments, the host cell has been transfected or transformed (e.g., by a recombinant polynucleotide of the present invention). In some embodiments, the host cell has been infected (e.g., by a viral particle of the present invention). In particular embodiments, the host cell comprises a plurality of recombinant polynucleotides and/or viral particles of the present invention. In some embodiments, the host cell comprises a plurality of different recombinant polynucleotides and/or viral particles of the present invention. In some embodiments, a viral particle of the present invention is replicating inside the host cell.

The host cell may be any cell of interest. The cell can be a cell from any organism, e.g., a bacterial cell, a cell of a single-cell eukaryotic organism, the cell of a multicellular eukaryotic organism, a plant cell (e.g., a rice cell, a wheat cell, a tomato cell, an Arabidopsis thaliana cell, a Zea mays cell and the like), an animal cell, a cell from an invertebrate animal (e.g., fruit fly, cnidarian, echinoderm, nematode, etc.), a cell from a vertebrate animal (e.g., fish, amphibian, reptile, bird, mammal, etc.), a cell from a mammal, a cell from a human, a cell from a healthy human, a cell from a human patient, a cell from a cancer patient, etc. In some cases, the host cell can be transplanted to a subject (e.g., patient). For instance, the cell can be derived from the subject to be treated (e.g., patient).

Furthermore, the cell can be a stem cell, e.g., embryonic stem cell, induced pluripotent stem cell, adult stem cell, e.g., mesenchymal stem cell, neural stem cell, hematopoietic stem cell, organ stem cell, a progenitor cell, a somatic cell, e.g., fibroblast, epithelial cell, endothelial cell, heart cell, liver cell, pancreatic cell, muscle cell, skin cell, blood cell, neural cell, immune cell, and any other cell of the body, e.g., human body. The cell can be a primary cell or a primary cell culture derived from a subject, e.g., an animal subject or a human subject, and allowed to grow in vitro for a limited number of passages. The cell can be a healthy cell or a diseased cell. In some embodiments, the host cell is a fibroblast (e.g., telomerized fibroblast). In particular embodiments, a recombinant polynucleotide of the present invention or viral particle of the present invention is purified from the host cell.

### General Recombinant Technology

Basic texts disclosing general methods and techniques in the field of recombinant genetics include Sambrook and Russell, Molecular Cloning, A Laboratory Manual (3rd ed. 2001); Kriegler, Gene Transfer and Expression: A Laboratory Manual (1990); and Ausubel et al., eds., Current Protocols in Molecular Biology (1994).

For nucleic acids, sizes are given in either kilobases (kb) or base pairs (bp). In some instances, these are estimates derived from agarose or acrylamide gel electrophoresis, from sequenced nucleic acids, or from published DNA sequences. For proteins, sizes are given in kilodaltons (kDa) or amino acid residue numbers. In some instances, protein sizes are estimated from gel electrophoresis, from sequenced proteins, from derived amino acid sequences, or from published protein sequences.

Oligonucleotides that are not commercially available can be chemically synthesized, e.g., according to the solid phase phosphoramidite triester method first described by Beaucage & Caruthers, Tetrahedron Lett. 22: 1859-1862 (1981), using an automated synthesizer, as described in Van Devanter et. al., Nucleic Acids Res. 12: 6159-6168 (1984). Purification of oligonucleotides is performed using any art-recognized strategy, e.g., native acrylamide gel electrophoresis or anion-exchange HPLC as described in Pearson & Reanier, J. Chrom. 255: 137-149 (1983).

The sequence of a protein domain or gene of interest can be verified after cloning or subcloning using, e.g., the chain termination method for sequencing double-stranded templates of Wallace et al., Gene 16: 21-26 (1981).

### Coding Sequence for a Protein of Interest

The present invention provides recombinant polynucleotides (e.g., isolated recombinant polynucleotides) that comprise a nucleic acid sequence encoding a protein of interest (e.g., an antigen, immunostimulatory protein, and/or selectable marker). The rapid progress in the studies of various genomes (e.g., the human genome) has made possible a cloning approach where a human or other model organism DNA sequence database can be searched for any gene segment that has a certain percentage of sequence homology to a known nucleotide sequence, such as one encoding an antigen, immunostimulatory protein, selectable marker, etc. Any DNA sequence so identified can be subsequently obtained by chemical synthesis and/or a polymerase chain reaction (PCR) technique such as overlap extension method. For a short sequence, completely de novo synthesis may be sufficient; whereas further isolation of full length coding sequence from a human or other model organism cDNA or genomic library using a synthetic probe may be necessary to obtain a larger gene.

Alternatively, a nucleic acid sequence can be isolated from a cDNA or genomic DNA library (e.g., human or rodent cDNA or human, rodent, bacterial, or viral genomic DNA library) using standard cloning techniques such as polymerase chain reaction (PCR), where homology-based primers can often be derived from a known nucleic acid sequence. Most commonly used techniques for this purpose are described in standard texts, e.g., Sambrook and Russell, supra.

cDNA libraries may be commercially available or can be constructed. The general methods of isolating mRNA, making cDNA by reverse transcription, ligating cDNA into a recombinant vector, transfecting into a recombinant host for propagation, screening, and cloning are well known (see, e.g., Gubler and Hoffman, Gene, 25: 263-269 (1983); Ausubel et al., supra). Upon obtaining an amplified segment of nucleotide sequence by PCR, the segment can be further used as a probe to isolate the full length polynucleotide sequence encoding the protein of interest from the cDNA library. A general description of appropriate procedures can be found in Sambrook and Russell, supra.

A similar procedure can be followed to obtain a full-length sequence encoding a protein of interest from a human or other model organism genomic library. Genomic libraries are commercially available or can be constructed according to various art-recognized methods. As a non-limiting example, to construct a genomic library, the DNA is first extracted from a tissue where a protein of interest is likely found. The DNA is then either mechanically sheared or enzymatically digested to yield fragments of about 12-20 kb in length. The fragments are subsequently separated by gradient centrifugation from polynucleotide fragments of undesired sizes and are inserted in bacteriophage λ vectors. These vectors and phages are packaged in vitro. Recombinant phages are analyzed by plaque hybridization as described in Benton and Davis, Science, 196: 180-182 (1977). Colony hybridization is carried out as described by Grunstein et al., Proc. Natl. Acad. Sci. USA, 72: 3961-3965 (1975).

Based on sequence homology, degenerate oligonucleotides can be designed as primer sets and PCR can be performed under suitable conditions (see, e.g., White et al., PCR Protocols: Current Methods and Applications, 1993; Griffin and Griffin, PCR Technology, CRC Press Inc. 1994) to amplify a segment of nucleotide sequence from a cDNA or genomic library. Using the amplified segment as a probe, the full-length nucleic acid encoding a protein of interest is obtained.

Upon acquiring a nucleic acid sequence encoding a protein of interest, the coding sequence can be further modified by a number of well-known techniques such as restriction endonuclease digestion, PCR, and PCR-related methods to generate coding sequences, including mutants and variants derived from the wild-type protein. The polynucleotide sequence encoding the desired polypeptide can then be subcloned into a vector, for instance, an expression vector, so that a recombinant polypeptide can be produced from the resulting construct. Further modifications to the coding sequence, e.g., nucleotide substitutions, may be subsequently made to alter the characteristics of the polypeptide.

A variety of mutation-generating protocols are established and described in the art, and can be readily used to modify a polynucleotide sequence encoding a protein of interest. See, e.g., Zhang et al., Proc. Natl. Acad. Sci. USA, 94: 4504-4509 (1997); and Stemmer, Nature, 370: 389-391 (1994). The procedures can be used separately or in combination to produce variants of a set of nucleic acids, and hence variants of encoded polypeptides. Kits for mutagenesis, library construction, and other diversity-generating methods are commercially available.

Mutational methods of generating diversity include, for example, site-directed mutagenesis (Botstein and Shortle, Science, 229: 1193-1201 (1985)), mutagenesis using uracil-containing templates (Kunkel, Proc. Natl. Acad. Sci. USA, 82: 488-492 (1985)), oligonucleotide-directed mutagenesis (Zoller and Smith, Nucl. Acids Res., 10: 6487-6500 (1982)), phosphorothioate-modified DNA mutagenesis (Taylor et al., Nucl. Acids Res., 13: 8749-8787 (1985)), and mutagenesis using gapped duplex DNA (Kramer et al., Nucl. Acids Res., 12: 9441-9456 (1984)).

Other possible methods for generating mutations include point mismatch repair (Kramer et al., Cell, 38: 879-887 (1984)), mutagenesis using repair-deficient host strains (Carter et al., Nucl. Acids Res., 13: 4431-4443 (1985)), deletion mutagenesis (Eghtedarzadeh and Henikoff, Nucl. Acids Res., 14: 5115 (1986)), restriction-selection and restriction-purification (Wells et al., Phil. Trans. R. Soc. Lond. A, 317: 415-423 (1986)), mutagenesis by total gene synthesis (Nambiar et al., Science, 223: 1299-1301 (1984)), double-strand break repair (Mandecki, Proc. Natl. Acad. Sci. USA, 83: 7177-7181 (1986)), mutagenesis by polynucleotide chain termination methods (U.S. Pat. No. 5,965,408), and error-prone PCR (Leung et al., Biotechniques, 1: 11-15 (1989)).

For recombinant modification of viral genomes or portions thereof (e.g., CMV genomes or portions thereof) in the construction of recombinant polynucleotides of the present invention, modification can be achieved, for example, using bacterial cells such as E. coli cells. As a non-limiting example, a bacterial cell comprising a bacterial artificial chromosome (BAC) that contains a CMV genome (or a portion thereof) of interest can be generated or obtained, and then the CMV genome or portion thereof can be recombinantly modified, for example using Red/ET recombination. Vectors and kits for performing Red/ET recombination are available from Gene Bridges and are described further in U.S. Pat. Nos. 6,355,412 and 6,509,156. Other suitable methods will also be known to one of skill in the art.

### IV. Methods for Inducing an Immune Response and Treating Disease

In another aspect, pharmaceutical compositions are provided. In some embodiments, the pharmaceutical composition comprises a recombinant polynucleotide of the present invention, a viral particle of the present invention (e.g., a viral particle comprising a recombinant polynucleotide of the present invention), and/or a host cell of the present invention (e.g., a host cell comprising a recombinant polynucleotide of the present invention and/or a viral particle of the present invention) and a pharmaceutically acceptable carrier.

In another aspect, methods for inducing an immune response against an antigen (e.g., in a subject) are provided. In some embodiments, the method comprises administering a recombinant polynucleotide (e.g., a therapeutically effective amount thereof) of the present invention to a subject (e.g., a subject in need thereof). In some embodiments, the method comprises administering a viral particle (e.g., a therapeutically effective amount thereof) of the present invention to a subject (e.g., a subject in need thereof). In some embodiments, the method comprises administering a host cell (e.g., a therapeutically effective amount thereof) of the present invention to a subject (e.g., a subject in need thereof). In some embodiments, the method comprises administering a pharmaceutical composition (e.g., a therapeutically effective amount thereof) of the present invention to a subject (e.g., a subject in need thereof).

The antigen against which an immune response is generated (e.g., in a subject) will depend on the particular disease(s) for which prophylactic and/or therapeutic benefit is sought. In some embodiments, the antigen is an infectious disease antigen. In other embodiments, the antigen is a tumor-associated antigen. In some embodiments, the antigen is both an infectious disease and a tumor-associated antigen. In particular embodiments, inducing an immune response against an infectious disease antigen prevents or treats a disease that is caused or exacerbated by the infectious disease. As a non-limiting example, inducing an immune response (e.g., in a subject) against an infectious disease antigen can prevent and/or treat a cancer that is caused or exacerbated by the particular infectious disease associated with that antigen. As another non-limiting example, inducing an immune response against an infectious disease that causes immunodeficiency (e.g., in a subject), such as HIV or SIV, can prevent and/or treat diseases that result from the immunodeficiency.

In some embodiments, an immune response (e.g., a desired, intended, or protective immune response, e.g., in a subject) is induced against a bacterial antigen (e.g., a bacterial infectious disease antigen). In some embodiments, an immune response is induced against a viral antigen (e.g., a viral infectious disease antigen). In some embodiments, an immune response is induced against a fungal antigen (e.g., a fungal infectious disease antigen). In some embodiments, an immune response is induced against a protozoal antigen (e.g., a protozoal infectious disease antigen). In some embodiments, an immune response is induced against a helminthic antigen (e.g., a helminthic infectious disease antigen). In some embodiments, the antigen is a bacterial, viral, fungal, protozoal, and/or helminthic antigen. In particular embodiments, the antigen is derived from a parasite.

In some instances, the antigen (e.g., infectious disease antigen) is from simian immunodeficiency virus (SIV). In some instances, the antigen is from human immunodeficiency virus (HIV). In some instances, the antigen is from hepatitis C virus. In some instances, the antigen is from a herpes simplex virus. In some instances, the antigen is from Epstein-Barr virus. In some instances, the antigen is from simian immunodeficiency virus (SIV), human immunodeficiency virus (HIV), hepatitis C virus, herpes simplex virus, Epstein-Barr virus, or a combination thereof. Non-limiting examples of suitable infectious disease antigens include SIV and HIV gag proteins. In some embodiments, the infectious disease antigen is a bacterial infectious disease antigen from Mycobacterium tuberculosis.

Compositions and methods of the present invention are useful for inducing a response (e.g., a desired, intended, or protective immune response) against any number of tumor-associated antigens (TAAs). The TAA can be derived from, for example, a colorectal cancer cell, a colon cancer cell, an anal cancer cell, a liver cancer cell, an ovarian cancer cell, a breast cancer cell, a lung cancer cell, a bladder cancer cell, a thyroid cancer cell, a pleural cancer cell, a pancreatic cancer cell, a cervical cancer cell, a prostate cancer cell, a testicular cancer cell, a bile duct cancer cell, a gastrointestinal carcinoid tumor cell, an esophageal cancer cell, a gall bladder cancer cell, a rectal cancer cell, an appendix cancer cell, a small intestine cancer cell, a stomach (gastric) cancer cell, a renal cancer (e.g., renal cell carcinoma) cell, a central nervous system cancer cell, a skin cancer cell, an oral squamous cell carcinoma cell, a choriocarcinoma cell, a head and neck cancer cell, a bone cancer cell, an osteogenic sarcoma cell, a fibrosarcoma cell, a neuroblastoma cell, a glioma cell, a melanoma cell, a leukemia (e.g., acute lymphocytic leukemia, chronic lymphocytic leukemia, acute myelogenous leukemia, chronic myelogenous leukemia, or hairy cell leukemia) cell, a lymphoma (e.g., non-Hodgkin's lymphoma, Hodgkin's lymphoma, B-cell lymphoma, or Burkitt's lymphoma) cell, a multiple myeloma cell, or any combination thereof. In particular embodiments, the TAA is derived from an ovarian cancer cell, a melanoma cell, a prostate cancer cell, or a combination thereof.

Non-limiting examples of TAAs to which an immune response (e.g., a desired, intended, or protective immune response) can be induced (e.g., using compositions and methods of the present invention) include the melanoma-associated antigens (MAGEs). MAGE proteins contain a conserved domain that is about 200 amino acids in length and is usually located near the C-terminal end of the protein, although the conserved domain is located closer to the central portion of some MAGE proteins. Human MAGE proteins include MAGEA1, MAGEA2, MAGEA2B, MAGEA3, MAGEA4, MAGEA5, MAGEA6, MAGEA7P, MAGEA8, MAGEA9, MAGEA9B, MAGEA10, MAGEA11, MAGEA12, MAGEA13P, MAGEB1, MAGEB2, MAGEB3, MAGEB4, MAGEB5, MAGEB6, MAGEB10, MAGEB16, MAGEB17, MAGEB18, MAGEC1, MAGEC2, MAGEC3, MAGED1, MAGED2, MAGED3 (also known as “trophin” or “TRO”), MAGED4, MAGED4B, MAGEE1, MAGEE2, MAGEF1, MAGEEG1 (also known as “NSMCE3”), MAGEH1, MAGEL2, and NDN. Additional non-limiting examples of TAAs that are useful for the present invention include NY-ESO-1 and prostate-specific antigen (PSA). In some embodiments, the TAA is a neoantigen.

In some embodiments, an immune response (e.g., a desired, intended, or protective immune response) is induced against an antigen selected from the group consisting of MAGEA4, MAGEA10, NY-ESO-1, PSA, and a combination thereof.

In some embodiments, the immune response (e.g., a desired, intended, or protective immune response) that is induced (e.g., in a subject) using a composition of the present invention (e.g., comprising a recombinant polynucleotide comprising a CMV genome or portion thereof and an antigen, wherein the CMV genome or portion thereof comprises one or more immunomodulatory mutations, wherein the one or more immunomodulatory mutations comprise a mutation within a nucleic acid sequence encoding a protein that has interleukin-10 (IL-10)-like activity) is greater than the immune response that is induced using a composition that does not comprise the mutation within the nucleic acid sequence encoding the protein that has IL-10-like activity. In some embodiments, compositions and methods of the present invention generate an increased inflammatory immune response.

In some embodiments, the induced response (e.g., desired, intended, or protective immune response) is increased by at least about 1.1-fold, 1.2-fold, 1.3-fold, 1.4-fold, 1.5-fold, 1.6-fold, 1.7-fold, 1.8-fold, 1.9-fold, 2-fold, 2.1-fold, 2.2-fold, 2.3-fold, 2.4-fold, 2.5-fold, 2.6-fold, 2.7-fold, 2.8-fold, 2.9-fold, 3-fold, 3.1-fold, 3.2-fold, 3.3-fold, 3.4-fold, 3.5-fold, 3.6-fold, 3.7-fold, 3.8-fold, 3.9-fold, 4-fold, 4.1-fold, 4.2-fold, 4.3-fold, 4.4-fold, 4.5-fold, 4.6-fold, 4.7-fold, 4.8-fold, 4.9-fold, 5-fold, 5.1-fold, 5.2-fold, 5.3-fold, 5.4-fold, 5.5-fold, 5.6-fold, 5.7-fold, 5.8-fold, 5.9-fold, 6-fold, 6.1-fold, 6.2-fold, 6.3-fold, 6.4-fold, 6.5-fold, 6.6-fold, 6.7-fold, 6.8-fold, 6.9-fold, 7-fold, 7.1-fold, 7.2-fold, 7.3-fold, 7.4-fold, 7.5-fold, 7.6-fold, 7.7-fold, 7.8-fold, 7.9-fold, 8-fold, 8.1-fold, 8.2-fold, 8.3-fold, 8.4-fold, 8.5-fold, 8.6-fold, 8.7-fold, 8.8-fold, 8.9-fold, 9-fold, 9.1-fold, 9.2-fold, 9.3-fold, 9.4-fold, 9.5-fold, 9.6-fold, 9.7-fold, 9.8-fold, 9.9-fold, 10-fold, 10.5-fold, 11-fold, 11.5-fold, 12-fold, 12.5-fold, 13-fold, 13.5-fold, 14-fold, 14.5-fold, 15-fold, 15.5-fold, 16-fold, 16.5-fold, 17-fold, 17.5-fold, 18-fold, 18.5-fold, 19-fold, 19.5-fold, 20-fold 21-fold, 22-fold, 23-fold, 24-fold, 25-fold, 26-fold, 27-fold, 28-fold, 29-fold, 30-fold, 31-fold, 32-fold, 33-fold, 34-fold, 35-fold, 36-fold, 37-fold, 38-fold, 39-fold, 40-fold, 41-fold, 42-fold, 43-fold, 44-fold, 45-fold, 46-fold, 47-fold, 48-fold, 49-fold, or 50-fold compared to the response that is induced using a composition that does not comprise the mutation (e.g., a mutation described herein or the mutation present in a particular embodiment of the present invention) within the nucleic acid sequence encoding the protein that has IL-10-like activity.

In some embodiments, inducing an immune response (e.g., desired, intended, or protective immune response, e.g., in a subject) comprises generating antibodies against an antigen (e.g., an antigen encoded by a recombinant polynucleotide or other compositions of the present invention). In some instances, antibodies are generated against an infectious disease antigen. In some instances, antibodies are generated against a TAA. In some instances, antibodies are generated against an infectious disease antigen and/or a TAA. In some embodiments, inducing an immune response comprises increasing the expression and/or activity of an immunostimulatory protein (e.g., in a subject). In some embodiments, inducing an immune response comprises increasing the expression and/or activity of a cytokine. In some instances, inducing an immune response comprises increasing the expression and/or activity of an interleukin (e.g., IL-12 and/or IL-15). In some embodiments, inducing an immune response comprises increasing the expression and/or activity of an interferon (e.g., interferon-gamma) and/or a tumor necrosis factor (e.g., tumor necrosis factor-alpha). In some embodiments, inducing an immune response comprises increasing the number and/or activation of one or more T cells (e.g., in a subject). Non-limiting examples include CD4⁺ T cells and/or MHC-E-restricted CD4⁺ and/or CD8⁺ T cells.

In yet another aspect, methods for preventing and/or treating diseases (e.g., in a subject) are provided. In some embodiments, the method comprises administering a recombinant polynucleotide (e.g., a therapeutically effective amount thereof) of the present invention to a subject (e.g., a subject in need thereof). In some embodiments, the method comprises administering a viral particle (e.g., a therapeutically effective amount thereof) of the present invention to a subject (e.g., a subject in need thereof). In some embodiments, the method comprises administering a host cell (e.g., a therapeutically effective amount thereof) of the present invention to a subject (e.g., a subject in need thereof). In some embodiments, the method comprises administering a pharmaceutical composition (e.g., a therapeutically effective amount thereof) of the present invention to a subject (e.g., a subject in need thereof).

Any number of diseases can be prevented and/or treated using compositions and/or methods of the present invention. In some embodiments, an infectious disease is prevented and/or treated. In other embodiments, cancer is prevented and/or treated. In some embodiments, an infectious disease and/or cancer are treated.

In some embodiments, a bacterial infectious disease is prevented and/or treated. In some embodiments, a viral infectious disease is prevented and/or treated. In some embodiments, a fungal infectious disease is prevented and/or treated. In some embodiments, a protozoal infectious disease is prevented and/or treated. In some embodiments, a helminthic infectious disease is prevented and/or treated. In some embodiments, a bacterial, viral, fungal, protozoal, and/or helminthic infectious disease is prevented and/or treated. In particular embodiments, the infectious disease is caused by a parasite. Non-limiting examples of viral infectious diseases that can be prevented and/or treated by the compositions and methods of the present invention include those caused by simian immunodeficiency virus (SIV), human immunodeficiency virus (HIV), hepatitis C virus, herpes simplex virus, and Epstein-Barr virus. A non-limiting example of a bacterial infectious disease that can be prevented and/or treated by the compositions and methods of the present invention is tuberculosis.

Non-limiting examples of cancers that can be prevented and/or treated using compositions and methods of the present invention include colorectal cancer, colon cancer, anal cancer, liver cancer, ovarian cancer, breast cancer, lung cancer, bladder cancer, thyroid cancer, pleural cancer, pancreatic cancer, cervical cancer, prostate cancer, testicular cancer, bile duct cancer, gastrointestinal carcinoid tumors, esophageal cancer, gall bladder cancer, rectal cancer, appendix cancer, small intestine cancer, stomach (gastric) cancer, renal cancer (e.g., renal cell carcinoma), cancer of the central nervous system, skin cancer, oral squamous cell carcinoma, choriocarcinomas, head and neck cancers, bone cancer, osteogenic sarcomas, fibrosarcoma, neuroblastoma, glioma, melanoma, leukemia (e.g., acute lymphocytic leukemia, chronic lymphocytic leukemia, acute myelogenous leukemia, chronic myelogenous leukemia, or hairy cell leukemia), lymphoma (e.g., non-Hodgkin's lymphoma, Hodgkin's lymphoma, B-cell lymphoma, or Burkitt's lymphoma), and multiple myeloma. In some embodiments, the cancer is melanoma, ovarian cancer, or prostate cancer.

Compositions and methods of the present invention can be used to treat cancer at any stage. In some embodiments, the cancer is an advanced cancer. In some embodiments, the cancer is a metastatic cancer. In some embodiments, the cancer is a drug-resistant cancer.

In some embodiments, the subject is treated (e.g., an immune response against an antigen is induced) before any symptoms or sequelae of the disease (e.g., infectious disease, or cancer) develop. In other embodiments, the subject has signs, symptoms, or sequelae of the disease. In some instances, treatment results in a reduction or elimination of the signs, symptoms, or sequelae of the disease.

In some embodiments, prevention and/or treatment includes administering compositions of the present invention directly to a subject. As a non-limiting example, pharmaceutical compositions of the present invention (e.g., comprising a recombinant polynucleotide, viral particle, and/or host cell of the present invention and a pharmaceutically acceptable carrier) can be delivered directly to a subject (e.g., by local injection or systemic administration). In some instances, intratumoral injection is used. In other embodiments, the compositions of the present invention are delivered to a host cell or population of host cells, and then the host cell or population of host cells is administered or transplanted into the subject. The host cell or population of host cells can be administered or transplanted with a pharmaceutically acceptable carrier. In certain instances, progeny of the host cell or population of host cells are transplanted into the subject. Procedures for transplantation and administration will be known to one of skill in the art.

Compositions of the present invention (e.g., recombinant polynucleotides, viral particles, host cells, and pharmaceutical compositions described herein) may be administered as a single dose or as multiple doses, for example two doses administered at a suitable interval. In some embodiments, the interval is about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days. In some embodiments, the interval is about 1, 2, 3, 4, 5, 6, 7, 8, 9, or 10 weeks. In some embodiments, the interval is about 1, 2, 3, 4, 5, 6, 7, 8, 9, 10, 11, or 12 months. When three or more doses are administered, the interval between any two doses can be the same, but do not need to be so. Other suitable dosage schedules can be determined by a medical practitioner.

In some embodiments, a dose comprises between about 10⁴ and about 10⁸ plaque-forming units (pfu) (e.g., about 10⁴, 10⁵, 10⁶, 10⁷, or 10⁸ pfu). In some embodiments, a dose comprises about 10⁴ pfu to about 10⁸ pfu, about 10⁴ pfu to about 10⁷ pfu, about 10⁴ pfu to about 10⁶ pfu, about 10⁴ pfu to about 10⁵ pfu, about 10⁵ pfu to about 10⁸ pfu, about 10⁵ pfu to about 10⁷ pfu, about 10⁵ pfu to about 10⁶ pfu, about 10⁶ pfu to about 10⁸ pfu, or about 10⁶ pfu to about 10⁷ pfu. In particular embodiments, a dose comprises between about 10⁴ and about 2×10⁷ pfu. The dose will vary depending on factors such as the particular antigen to which an immune response is being induced, characteristics of the recombinant polynucleotide encoding the antigen, immune status of the subject, age of the subject, weight of the subject, concomitant medical conditions, route of administration, etc.

In some embodiments, additional compounds or medications can be co-administered to the subject. Such compounds or medications can be co-administered for the purpose of alleviating signs or symptoms of the disease being treated, reducing side effects cause by induction of the immune response, etc.

In some embodiments, methods of the present invention (e.g., for inducing an immune response or for preventing or treating a disease) comprise increasing or decreasing the unfolded protein response (UPR). In some instances, the UPR is increased (e.g., in a subject). In other instances, the UPR is decreased (e.g., in a subject). In some instances, the ability of a CMV to inhibit the UPR in decreased. In other instances, the ability of CMV to inhibit the UPR is increased. In some instances, the ability of CMV to inhibit the UPR is decreased by introducing mutations such as deletions or substitutions into nucleic acid sequences encoding, e.g., Human cytomegalovirus UL50, Rhesus cytomegalovirus Rh81, Mouse cytomegalovirus M50, or homologs thereof. In other instances, the ability of CMV to inhibit the UPR is increased by increasing the expression or activity of, e.g., Human cytomegalovirus UL50, Rhesus cytomegalovirus Rh81, Mouse cytomegalovirus M50, or homologs thereof. Expression or activity can be increased, for example, by placing the nucleic acid sequences encoding these proteins under the control of an appropriate promoter. Inhibiting the UPR (e.g., by increasing the ability of CMV to suppress the UPR) can be desirable, for example, in a tumor microenvironment (e.g., when delivering a composition of the present invention by intratumoral injection).

In some embodiments, a sample (e.g., a test sample) is obtained from a subject (e.g., a subject in whom an immune response against an antigen is to be induced or a subject in whom a disease is to be prevented and/or treated). In particular embodiments, the sample is obtained for the purposes of determining the presence or level of one or biomarkers. Determining the presence or level of biomarkers(s) (e.g., in a sample) can be used to, as non-limiting examples, determine response to treatment or to select an appropriate composition or method for the prevention or treatment of a disease.

In particular embodiments, a test sample is obtained from the subject. The test sample can be obtained before and/or after a composition of the present invention is administered to the subject. Non-limiting examples of suitable samples include blood, serum, plasma, cerebrospinal fluid (CSF), tissue, saliva, urine, and combinations thereof. In some instances, the sample comprises normal tissue. In other instances, the sample comprises abnormal tissue (e.g., cancer tissue). The sample can also be made up of a combination of normal and abnormal cells (e.g., cancer cells). In some instances, the sample is obtained as a biopsy sample or fine needle aspirate (FNA) sample. In some embodiments, the tissue comprises one or more types of immune cells.

In some embodiments, a reference sample is obtained. The reference sample can be obtained, for example, from the subject (i.e., the subject being treated or in whom an immune response is being induced). The reference sample can be also be obtained from a different subject and/or a population of subjects. In some instances, the reference sample is either obtained from the subject, a different subject, or a population of subjects before and/or after a composition of the present invention is administered to the subject, and comprises normal tissue. In other instances, the reference sample comprises abnormal tissue and is obtained from the subject and/or from a different subject or a population of subjects.

In some embodiments, the level of one or more biomarkers is determined in the test sample and/or reference sample. Non-limiting examples of suitable biomarkers include antigens, antibodies against antigens, immune cell numbers and/or activation levels, capacity for immune cell responses to an antigen after in vitro stimulation, immunostimulatory proteins, cytokines, interleukins, tumor necrosis factors, interferons, and other molecules that play roles in modulating immune responses. Further non-limiting examples of suitable biomarkers include C-reactive protein, interferon-gamma, IL-4, IL-5, IL-6, IL-10, IL-12, IL-15, tumor necrosis factor-alpha, and combinations thereof.

Typically, the level of a biomarker in a sample (e.g., test sample) is compared to the level of the biomarker in a reference sample. Depending on the biomarker, an increase or a decrease relative to a normal control or reference sample can be indicative of the presence of a disease, or response to treatment for a disease. The difference between the reference sample or value and the test sample need only be sufficient to be detected. In some embodiments, an increased level of a biomarker in a sample (e.g., test sample), and hence the presence of a disease (e.g., an infectious disease or cancer), increased risk of the disease, or response to treatment is determined when the biomarker levels are at least, e.g., about 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, or 20-fold, higher in comparison to a negative control. In other embodiments, a decreased level of a biomarker in the test sample, and hence the presence of the disease, increased risk of the disease, or response to treatment is determined when the biomarker levels are at least, e.g., about 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, or 20-fold lower in comparison to a negative control.

The biomarker levels can be detected using any method known in the art, including the use of antibodies specific for the biomarkers. Exemplary methods include, without limitation, PCR, Western Blot, dot blot, enzyme-linked immunosorbent assay (ELISA), radioimmunoassay (RIA), immunoprecipitation, immunofluorescence, FACS analysis, electrochemiluminescence, and multiplex bead assays (e.g., using Luminex or fluorescent microbeads). In some instances, nucleic acid sequencing is employed.

In certain embodiments, the presence of decreased or increased levels of one or more biomarkers is indicated by a detectable signal (e.g., a blot, fluorescence, chemiluminescence, color, radioactivity) in an immunoassay or PCR reaction (e.g., quantitative PCR). This detectable signal can be compared to the signal from a control sample or to a threshold value.

In some embodiments, the results of the biomarker level determinations are recorded in a tangible medium. For example, the results of diagnostic assays (e.g., the observation of the presence or decreased or increased presence of one or more biomarkers) and the diagnosis of whether or not there is an increased risk or the presence of a disease (e.g., an infectious disease or cancer) or whether or not a subject is responding to treatment can be recorded, e.g., on paper or on electronic media (e.g., audio tape, a computer disk, a CD, a flash drive, etc.).

In other embodiments, the methods further comprise the step of providing the diagnosis to the patient (i.e., the subject) and/or the results of treatment.

### V. Kits

In another aspect, the present invention provides kits. In some embodiments, the kit comprises a recombinant polynucleotide of the present invention, a viral particle of the present invention (e.g., a viral particle comprising a recombinant polynucleotide of the present invention), a host cell of the present invention (e.g., a host cell comprising a recombinant polynucleotide of the present invention and/or a viral particle of the present invention), and/or a pharmaceutical composition of the present invention (e.g., comprising a recombinant polynucleotide of the present invention, viral particle of the present invention, and/or a host cell of the present invention and a pharmaceutically acceptable carrier). In some embodiments, the kit is for inducing an immune response against an antigen (e.g., in a subject). In other embodiments, the kit is for preventing or treating a disease (e.g., in a subject). In particular embodiments, the kit is for preventing or treating an infectious disease described herein and/or a cancer described herein.

Kits of the present invention can be packaged in a way that allows for safe or convenient storage or use (e.g., in a box or other container having a lid). Typically, kits of the present invention include one or more containers, each container storing a particular kit component such as a reagent, a control sample, and so on. The choice of container will depend on the particular form of its contents, e.g., a kit component that is in liquid form, powder form, etc. Furthermore, containers can be made of materials that are designed to maximize the shelf-life of the kit components. As a non-limiting example, kit components that are light-sensitive can be stored in containers that are opaque.

In some embodiments, the kit contains one or more reagents. In some instances, the reagents are useful for transfecting or transforming a host cell with a recombinant polynucleotide of the present invention. The kit may also comprise one or more reagents useful for delivering recombinant polynucleotides or viral particles of the present invention into a host cell and/or for administering a pharmaceutical composition of the present invention to a subject. In yet other embodiments, the kit further comprises instructions for use.

### VI. Examples

The present invention will be described in greater detail by way of specific examples. The following examples are offered for illustrative purposes only, and are not intended to limit the invention in any manner. Those of skill in the art will readily recognize a variety of noncritical parameters that can be changed or modified to yield essentially the same results.

### Example 1. RhCMVΔIL-10 Vectors

This example describes the construction of vectors of the present invention in which the viral IL-10 gene has been inactivated by a deletion within the viral IL-10 gene.

### Construction and Testing of RhCMVΔIL-10-Gag Vector

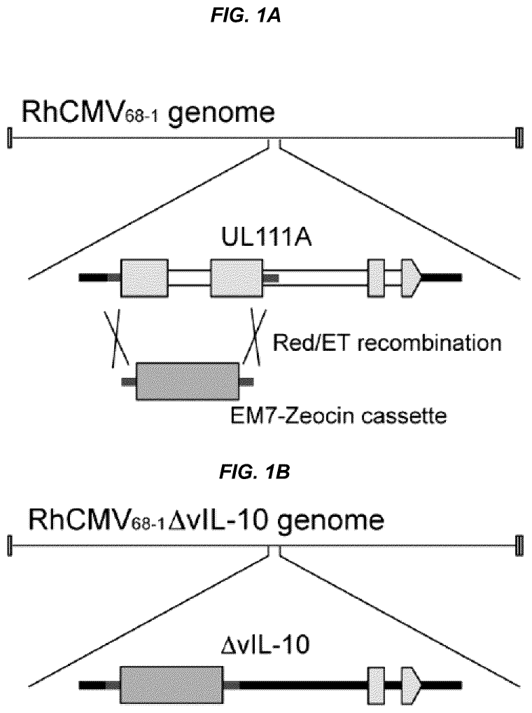

This vector was based upon a modified rhesus macaque CMV (RhCMV) genome. In order to construct the RhCMVΔIL-10-gag vector, the first two exons of the sequence encoding viral IL-10 were deleted from the parent genome (i.e., a BAC-cloned RhCMV68-1 genome (GenBank accession number JQ795930)), and replaced by an EM7-Zeocin expression cassette (SEQ ID NO:3). This is depicted in FIG. 1. As a result, the vector could not express the immunomodulatory protein, viral IL-10.

### Construction of the Vector

The full-length RhCMV BAC plasmid was mutated by ET recombination in Escherichia coli using the Red/ET Subcloning Kit (Gene Bridges, Germany). Briefly, plasmid pSC101-BAD-gbaA was transformed into E. coli DH10B containing the parental RhCMV68-1 BAC plasmid pRhCMV/BAC-Cre. The Red/ET proficient bacteria were generated by 0.1-0.2% L-arabinose induction. An EM7 promoter-controlled Zeocin gene cassette was amplified from pEM7/Zeo (Invitrogen) by PCR using a primer pair having the sequences set forth in SEQ ID NOS:4 and 5. These primers provided 50 nucleotides of viral sequences at their 5′ ends (SEQ ID NOS:1 and 2) that were required for homologous recombination between the PCR fragment and the first two exons of the RhCMV UL111A ORF within pRhCMV/BAC-Cre. The 550-bp PCR fragment was purified and introduced into Red/ET proficient E. coli by electroporation. The recombinant clones were selected on agar plates containing chloramphenicol (25 μg/mL) and Zeocin (25 μg/mL) at 37° C. Mutated RhCMV BAC plasmids were screened by PCR using primers having the sequences set forth in SEQ ID NOS:6 and 7.

Successfully mutated BAC plasmids were transfected into Telo-RF cells to reconstitute mutant viruses. The genome of mutated RhCMV was further analyzed by digestion with four different restriction enzymes, after separation of the DNA fragments on a 0.8% agarose gel and staining with ethidium bromide.

As described in Chang et al. PNAS (2010) 107:22647-22652, the IL-10-deleted virus infects seronegative rhesus monkeys and infection with this virus leads to greater cellularity at the site of infection as well as enhanced B cell and T cell responses.

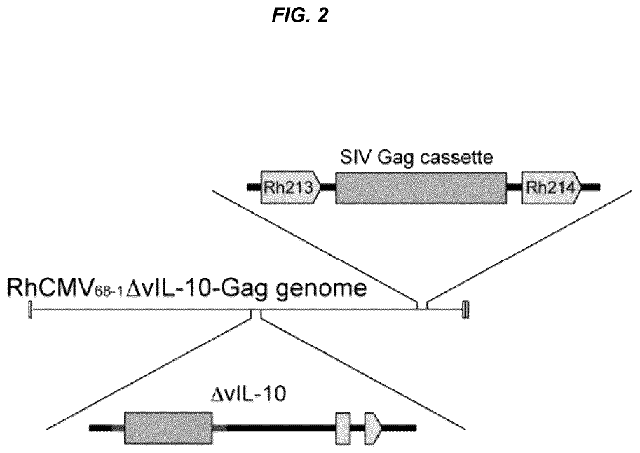

Insertion of an expression cassette for codon-optimized SIV gag is depicted in FIG. 2. The expression cassette was placed into the intergenic region between the Rh213 and Rh214 coding sequences. SIV gag fused with the Flag epitope tag (SEQ ID NO:9) was placed under the control of an EF1α promoter (SEQ ID NO:8) and followed by an SV40-derived polyadenylation site (SEQ ID NO:10).

### Testing of the Vector

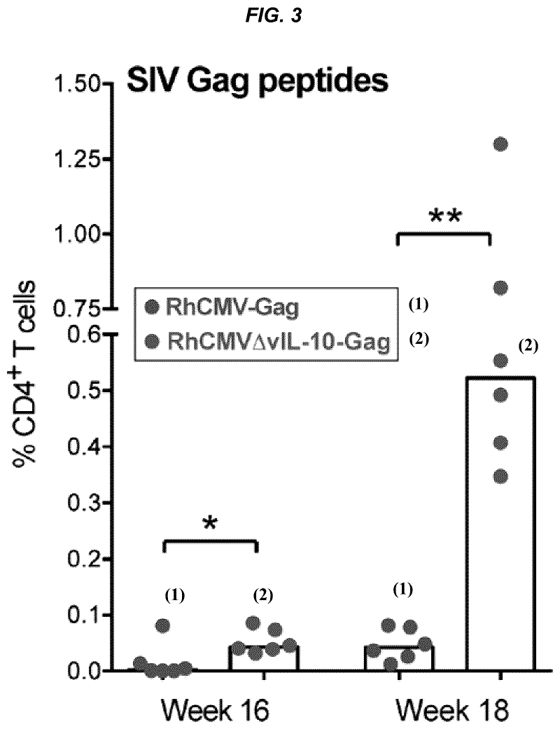

As shown in FIG. 3, immunization against SIV gag with the IL-10-deleted RhCMV vector induced significantly higher T cell responses against the vaccine target. PBMCs isolated before vaccine boost (i.e., at week 16) and after vaccine boost (i.e., at week 18) were stimulated with SIV gag overlapping peptides. Antigen-specific T cells were identified by co-expression of TNF-alpha and IFN-gamma (IFN-γ⁺TNF-α⁺) and presented as the percentage of total gated CD4⁺ T cells. Each data point in FIG. 3 represents an individual animal, and the bars represent mean values. Statistical analyses were performed using the Mann-Whitney nonparametric test.

### Additional RhCMVΔIL-10 Vectors

### RhCMVΔIL-10-MAGE-A4

This vector is identical to RhCMVΔIL-10-gag, except that it contains sequences directing expression of the human MAGE-A4 protein, a tumor-associated antigen (see, e.g., De Plaen et al. Immunogenetics (1994) 40:360-369 and Lurquin et al. Genomics (1997) 46:397-408).

### RhCMVΔIL-10-MAGE-A10

This vector is identical to RhCMVΔIL-10-gag, except that it contains sequences directing expression of the human MAGE-A10 protein, a tumor-associated antigen (see, e.g., Huang et al. J. Immunol. (1999) 162:6849-6854; De Plaen et al. Immunogenetics (1994) and Lurquin et al. Genomics (1997) 46:397-408).

RhCMV+Rh157.4truncΔIL-10-MAGE-A4

This vector is identical to RhCMVΔIL-10-MAGE-A4, except that it overexpresses the truncated Rh157.4 transcript found in the RhCMV strain 68-1 genome under control of the CAG promoter.

### RhCMV-HIL-12AIL-10-MAGE-A4

This vector is identical to RhCMVΔIL-10-MAGE-A4, except that it also expresses human IL-12 under control of the CAG promoter, so as to further tilt the cytokine environment toward Th1 responses.

### RhCMVΔIL-10ΔRh189-MAGE-A4

This vector is identical to RhCMVΔIL-10-MAGE-A4, except that it also contains a mutation in the sequence encoding Rh189 (i.e., a homolog of the human-specific CMV protein US11). The Rh189/US11 protein is a delayed-early gene whose product redirects nascent MHC class I proteins from the ER into the cytosol in a pattern similar to the misfolded protein response.

### RhCMVΔIL-10_repaired-MAGE-A4

This vector is identical to RhCMVΔIL-10-MAGE-A4, except that the deletion found in RhCMV strain 68-1 is repaired so that the coding sequence of this vector is identical to wild-type RhCMV. That is, expression of the Rh61/Rh60, Rh157.4, Rh157.5, and Rh157.6 genes will be restored (see, e.g., Lija et al. PNAS (2008) 105:19950-19955 and Malouli et al. J. Virol. (2012) 86:8959-8973).

### RhCMV+Rh189AIL-10-MAGE-A4

This vector is identical to RhCMVΔIL-10-MAGE-A4, except that it overexpresses Rh189 under the control of a strong synthetic promoter/enhancer such as the chicken beta-actin promoter coupled with the CMV early enhancer (i.e., the “CAG promoter”).

### RhCMVΔIL-10-CD40L-MAGE-A4

This vector is identical to RhCMVΔIL-10-MAGE-A4, except that it also expresses human or rhesus macaque CD40L (CD40 ligand; CD154) under control of the CAG promoter, so that CD40L appears on the surface of the produced RhCMV virions, allowing them to more easily interact with and infect dendritic cells.

In addition, any of the vectors described herein can be constructed using the genome of a CMV other than rhesus macaque CMV. As a non-limiting example, the vectors described herein can be constructed using human CMV.

### Example 2. Magnitude and Character of Response to RhCMVΔIL-10/SIV Gag Vaccine

This example describes a study designed to test the magnitude and character of immune responses to a viral IL-10-deleted RhCMV/SIV group-specific antigen (gag) vaccine.

### Experimental Approach

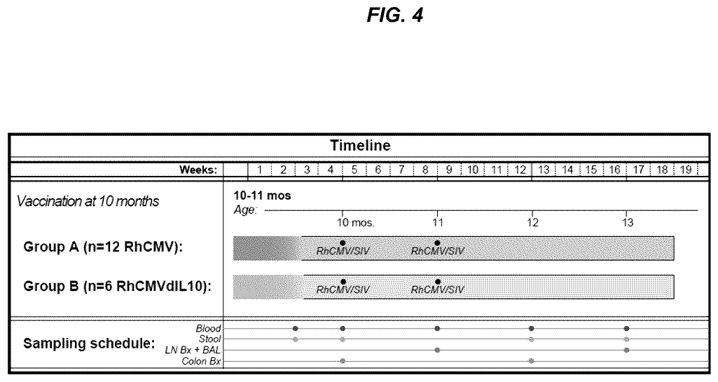

A timeline of the study is shown in FIG. 4. 12 infant rhesus macaques (8-10 months old) were immunized with conventional RhCMV/SIV gag and 6 infant macaques with RhCMV/SIV gag vaccine containing a deletion of the viral IL-10 gene (RhCMVΔIL-10/SIV gag). RhCMV/SIV gag was administered to (i) animals previously uninfected with wild-type RhCMV (conventional (non-SPF) animals screened seronegative; n=6) or (ii) animals previously infected with wild-type RhCMV (conventional and seropositive; n=6). RhCMVΔIL-10/SIV gag was administered only to animals previously uninfected with wild-type RhCMV (conventional and seronegative; n=6). Vaccine responses were then measured in parallel. It was hypothesized that immune responses provoked by RhCMVΔIL-10/SIV gag vaccine would be of greater magnitude and/or of different character (e.g., different cytokine or MHC restriction profile).

### RhCMV-SIV Vaccine Administration.

RhCMV/SIV gag and RhCMVΔIL-10/SIV gag vectors were produced and used in vaccines that were administered subcutaneously at a dose of 10⁵ plaque forming units (pfu) per vector, per administration, i.e., at both priming and boosting immunizations. All animals received priming vaccination at approximately 10 months of age.

Blood was drawn from all animals at 9.5, 10, 11, 12, and 13 months of age as well as more frequently when required for immunologic assays. The volume drawn did not exceed 12 mL/kg/month. Stool samples were collected from the cage pan in the morning and immediately frozen at −70° C. for later DNA extraction. Animals were briefly separated if necessary to ensure correctly identified stool samples. Lymph node biopsies and bronchioalveolar lavage (BAL) were performed by very experienced staff. For colon biopsies, animals were sedated with ketamine and dexmedetomidine. A lubricated endoscope was placed into the rectum and slowly advanced to the descending colon. Oval biopsy forceps were then advanced into the scope. For each animal, depending on the size, a few to a maximum of ten pinch biopsies were collected. Animals were treated with ketoprofen for 3 days afterward.

### Immunologic Profiling.

Phenotypic and functional characteristics of immune cells were assessed by flow cytometry using antibody panels (as in ref 1). For example, aliquots of cells were stained with four flow cytometry panels organized roughly around various phenotypes relevant to antigen-presenting, B, T, NK, and NKT cells. Additional aliquots were maintained in complete medium either (i) without stimulation or (ii) with stimulation by PMA and ionomycin. Still other aliquots were incubated with peptides from the gag protein to test responsiveness to the vaccine antigen. After overnight incubation, these latter aliquots were stained with a fifth panel containing antibodies specific for various cytokines relevant to T cell differentiation and cytokine production.

The quantity and quality of virus-specific T cell responses were assessed by cytokine flow cytometry. Assay wells containing up to one million peripheral blood mononuclear cells (PBMCs), lymph node mononuclear cells, or BAL cells were left unstimulated or stimulated with overlapping SIV gag peptides, RhCMV antigen, or PMA/ionomycin (i.e., serving as a positive control). All wells also received anti-CD28 and anti-CD49d at a concentration of 2 μg/mL. GolgiPlug (BD Biosciences) was added one hour after the start of incubation. Five hours later, samples were harvested by centrifugation, fixed, permeabilized, and stained using fixable live-dead stain as well as antibodies reactive to CD3, CD4, CD8, CD27, CD45RA, IL-2, IL-17, IFN-γ, and TNF-α. The fractions of cytokine-secreting CD4⁺ and CD8⁺ T cells were determined by flow cytometry on a Fortessa.

### Results

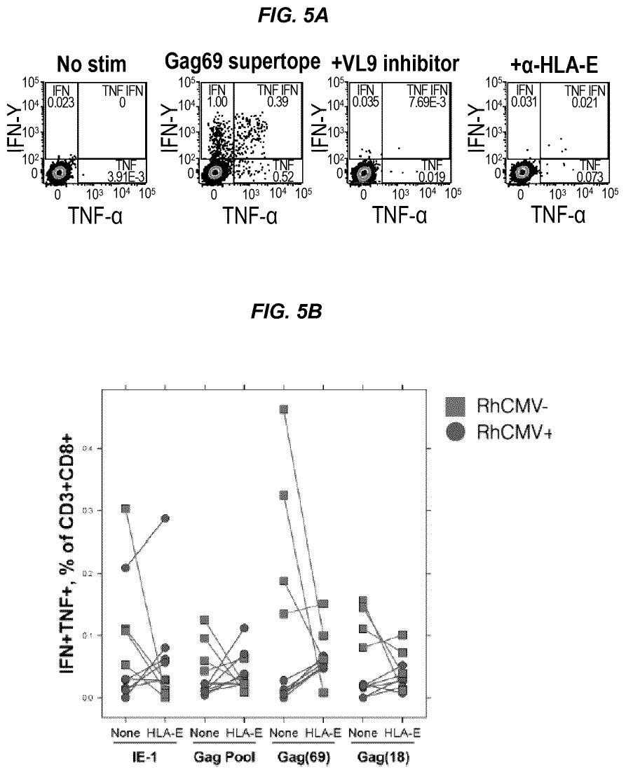

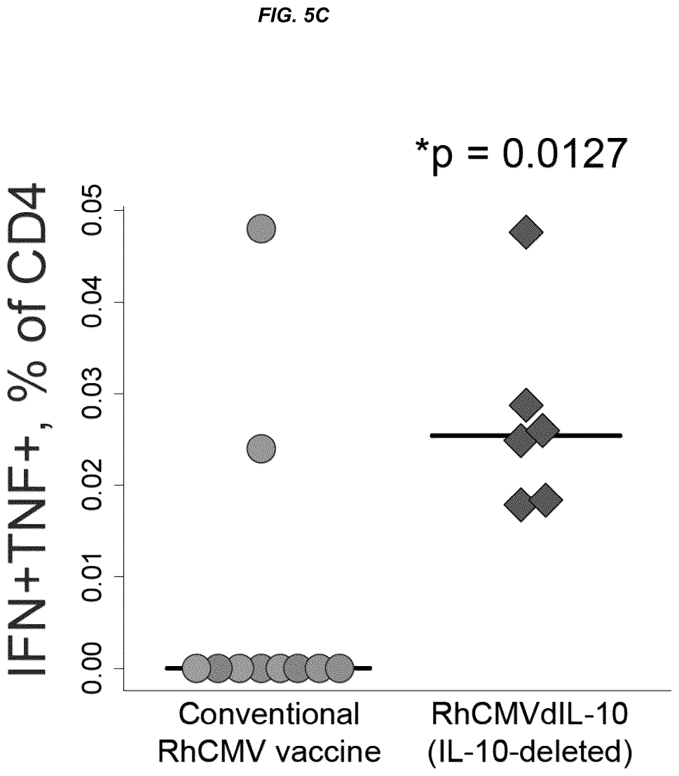

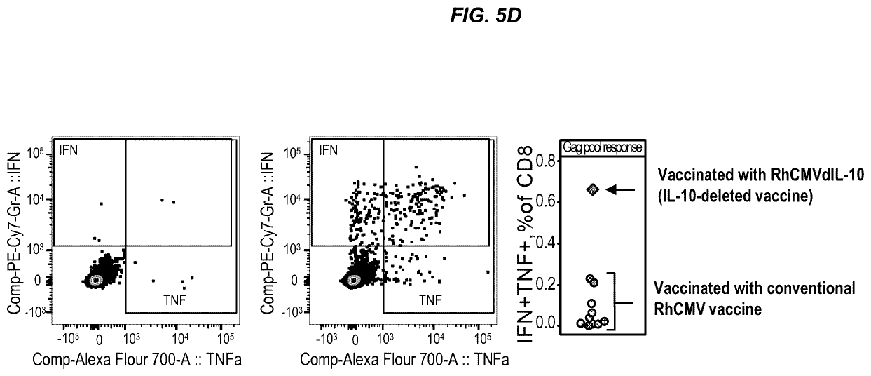

The RhCMV/SIV gag vaccine provoked T cell responses that appeared to be Mamu-E restricted. In particular, responses to the gag-69 peptide were observed among animals of unrelated Mamu types (FIGS. 5A and 5B). This peptide has previously been shown to be presented by the (shared) Mamu-E molecule, explaining the responses observed in MHC diverse animals. Furthermore, responses to this peptide were prevented by blocking with a tightly Mamu-E-binding peptide, VL9, presumably due to displacement of gag-69 from the Mamu-E molecule (FIG. 5A). Responses to gag-69 were also blocked in many instances by an anti-HLA-E antibody, which also binds to Mamu-E, disrupting the interaction of Mamu-E:peptide complex with the T cell receptor (FIG. 5B). T cell responses to vaccine were initially stronger in the wild-type RhCMV-negative group, but this difference between groups dissipated later in the experiment.

T cell responses were also tested in animals receiving RhCMVΔIL-10/SIV gag vaccine (FIGS. 5C and 5D). Two characteristics of immune responses to this vaccine were different, as compared to responses to RhCMV/SIV gag. First, an unusual consistency and higher average intensity of T cell responses to SIV gag peptides in the CD4⁺ T cell compartment were observed (FIG. 5C). Second, one animal manifested unusually strong responses among CD8⁺ T cells (FIG. 5D), which were stronger than any responses previously observed after administration of the original vaccine, i.e., with viral IL-10 intact. Thus, the hypotheses about the IL-10-deleted vaccine were confirmed. As compared to first-generation RhCMV/SIV gag vaccine, the IL-10-deleted vaccine provoked responses that were both higher in magnitude and of different character.

### Literature Cited

1. Ardeshir, A., N. R. Narayan, G. Mendez-Lagares, D. Lu, M. Rauch, Y. Huang, K. K. Van Rompay, S. V. Lynch, and D. J. Hartigan-O'Connor. Sci. Transl. Med., 2014. 6 (252): p. 252ra120.

### Example 3. Superior Protective Efficacy of RhCMVΔIL-10/SIV Gag Vaccine

An experiment was performed to test the efficacy of a viral IL-10-deficient rhesus CMV-based vaccine in protecting young macaques against high simian immunodeficiency virus (SIV) viremia, as compared to the efficacy of a previous vaccine.

Young macaques (8-11 months old) were assigned to the following groups: (A) 4 animals receiving no vaccine, (B) 12 animals receiving conventional RhCMV/SIV gag vaccine (i.e., with an intact viral IL-10 gene), and (C) 6 previously wild-type RhCMV-seronegative animals receiving RhCMVdIL10/SIVgag vaccine (i.e., lacking viral IL-10).

All animals were administered SIV vaccine at a dose of 10⁵ pfu subcutaneously, in no more than 2 mL volume at the start of the study. All animals then received a booster dose approximately 4 weeks after the first dose. Finally, beginning >16 weeks after the first vaccine dose, all animals were challenged with increasing doses of SIV. The challenges were administered every two weeks, orally, in a maximum volume of 2.5 mL. One week after each challenge, blood samples were drawn and assayed using sensitive PCR-based techniques for the presence of SIV. When infection was detected in a blood sample, challenges were discontinued for that animal. Blood samples were subsequently drawn approximately every week and viral loads tested, to determine how many animals in each group were able to control viremia, which is an indicator of successful vaccine-mediated protection.

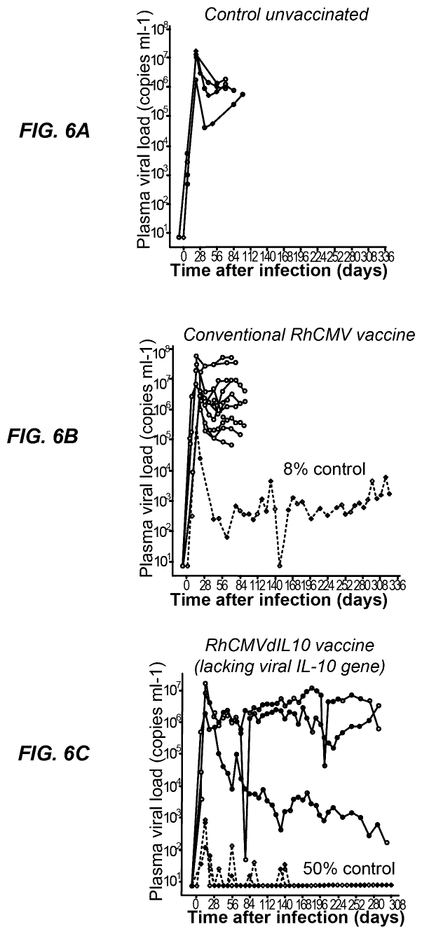

The results are shown in FIG. 6. Young rhesus macaques not receiving vaccine exhibited robust SIV infection, in accord with previous experience (FIG. 6A). Among animals receiving a conventional RhCMV/SIVgag vaccine, only 1 out of 12 was able to control infection (FIG. 6B). SIV copies in this animal's plasma were maintained between about 10² and 10³ per mL during the first several months after infection. Among animals receiving the viral IL-10-deficient vaccine of the present invention, however, 3 out of 6 animals (50%) were able to stringently control infection (FIG. 6C). Furthermore, these three animals were able to suppress viremia more robustly, frequently achieving levels below 100 copies per mL in the first month after infection.

### Example 4. Clearance of SIV Virus by Rhesus Macaques Receiving RhCMVΔIL-Vaccine

Macaques that are successfully protected against SIV by RhCMVΔIL-10/SIVgag vaccine often transiently manifest a small amount of virus in blood soon after SIV challenge, which then disappears and seems to have been cleared from the body (FIG. 6C). We performed two experiments to confirm that the virus was indeed cleared.

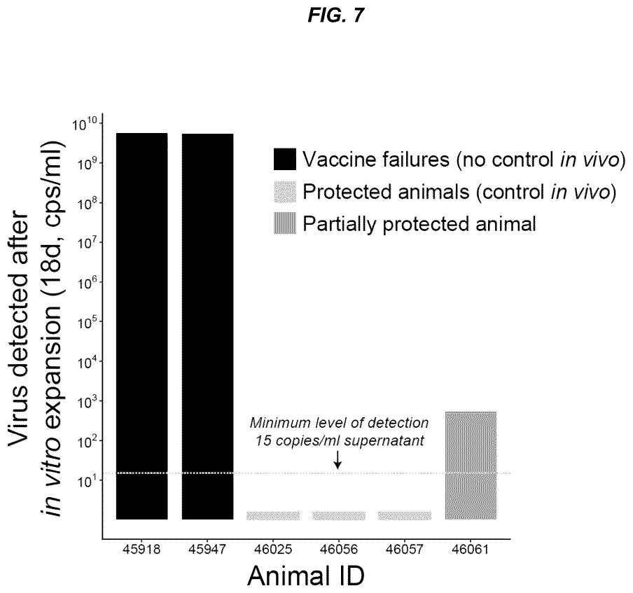

First, we sought to amplify any residual virus in peripheral blood cells by co-incubation of those cells with CEMx174 cells, which are known to provide an excellent substrate for SIV growth. In this assay, any rare virions with growth potential should interact with the CEMx174 cells and replicate, expanding over time until they are detectable. To perform the assay, we mixed 6×10⁶ CEMx174 cells in logarithmic growth phase with an equivalent number of PBMC from three animals that had been protected from infection by RhCMVΔIL-10/SIVgag vaccine, another animal with a low viral load, and finally two animals with a high viral load. This mixture of cells was incubated together for 18 days with addition of fresh media every 3-4 days to ensure continued robust cell growth. On day 18 the supernatants were assayed by real-time PCR. None of the three animals with undetectable viral loads yielded virus in this assay (i.e., the virus was not detectable after 18 days of co-culture), the animal with a low viral load gave a clear signal (580 copies per ml after 18 days), and those with high viral loads yielded correspondingly high levels of virus in the assay (FIG. 7).

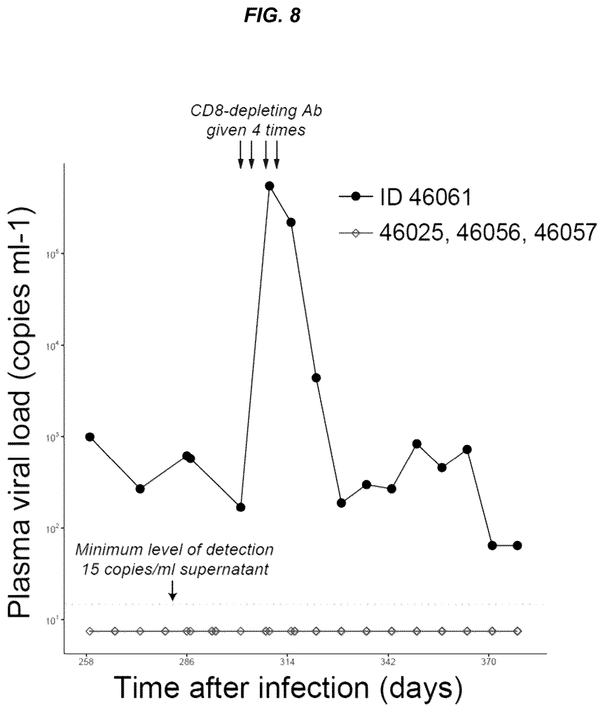

Second, we used anti-CD8 antibody to deplete CD8⁺ T cells from the vaccinated macaques. Because these cells contribute to control over SIV and HIV infections, their depletion encourages the virus to replicate. Our expectation was that animals with a detectable infection would manifest a higher viral load after CD8⁺ T cells were lost. Animals that have truly cleared the infection, on the other hand, were expected to continue to be free of circulating virus in peripheral blood. These predictions were met in the four animals given CD8⁺ T-cell depletion: virus was not detected in any of the three animals protected by vaccination, but the virus expanded in the single depleted animal known to be infected (FIG. 8).

### Example 5. Therapeutic Effect of RhcmvΔIL-10/SIVgag Vaccine Among Previously Infected Animals Receiving Antiretroviral Therapy

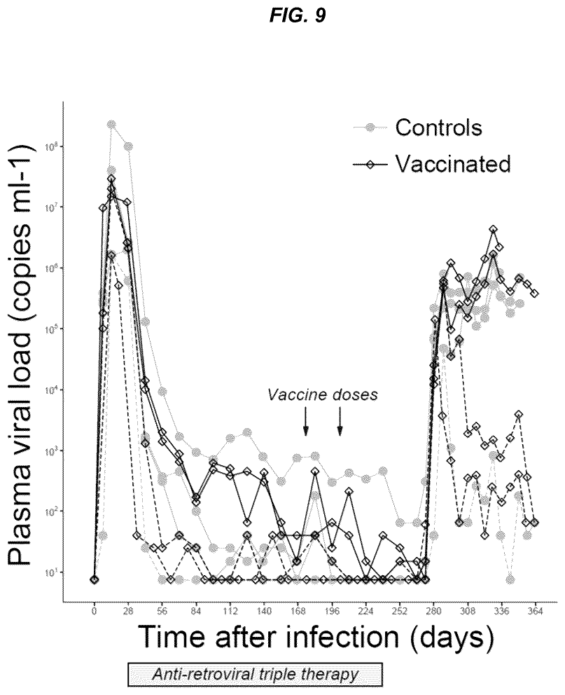

In this experiment we wanted to examine the therapeutic effect of viral IL-10-deficient RhCMV-vectored SIV vaccines against an established infection with the HIV model virus, SIV. Eight rhesus macaques were infected with SIVmac251 intrarectally (FIG. 9). Four weeks after infection, the macaques were given pharmacologic “triple therapy” (tenofovir, emtricitabine, and dolutegravir) to suppress the amount of virus in blood. This pharmacologic therapy was maintained for 35 weeks. During this 35-week period, four animals out of the eight animals were given two doses of RhCMVΔIL-10/SIVgag at weeks 21 and 25 after beginning triple therapy (25 and 29 weeks after SIV infection, respectively). Finally, the pharmacologic therapy was removed. The expectation was that, absent vaccination, virus would rebound after withdrawal of pharmacologic therapy, as the virus began to replicate without restraint by antiretroviral drugs.

The outcome of the experiment was: five of the eight infected animals rebounded as expected after withdrawal of therapy, while three demonstrated unusually low viral loads, presumably due to immunologic control (FIG. 9). One of these animals was in the control (unvaccinated) group, demonstrating a rate of immunologic control of 25% in that group. Two of these animals with unusually low viral loads were in the vaccinated group that received RhCMVΔIL-10/SIVgag, demonstrating that vaccination raised the rate of immunologic control to 50%.

### Example 6. Creation of RhCMVΔIL-10/MAGEA4 and RhCMVΔIL-10/MAGEA10 Vaccines

RhCMVΔIL-10/MAGEA4 and RhCMVΔIL-10/MAGEA10 vaccines were created as bacterial artificial chromosomes (BACs) in E. coli, then “rescued” as replicating vectors in rhesus telomerized fibroblasts.

The BACs were prepared by recombination of two DNA substrates in E. coli expressing the Red/ET recombination proteins. The first DNA substrate was RhCMV68-1AIL-BAC, carrying a deletion and replacement of the first two exons of the viral IL-10 gene. The second DNA substrate was a PCR product carrying an EF1alpha-MAGEA4-SV40 pA (or MAGEA10) cassette and a kanamycin-resistance gene flanked by FRT sites, prepared using PCR primers with extensions homologous to RhCMV sequences near the junction of the viral Rh213 and Rh214 genes. Recombination between these two DNA substrates resulted in insertion of the EF1alpha-MAGEA4-SV40 pA (or MAGEA10) and FRT-kan^{R}-FRT sequences between the Rh213 and Rh214 genes. Subsequent growth of this BAC in bacteria expressing the Flp recombinase resulted in removal of the kanamycin resistance gene.

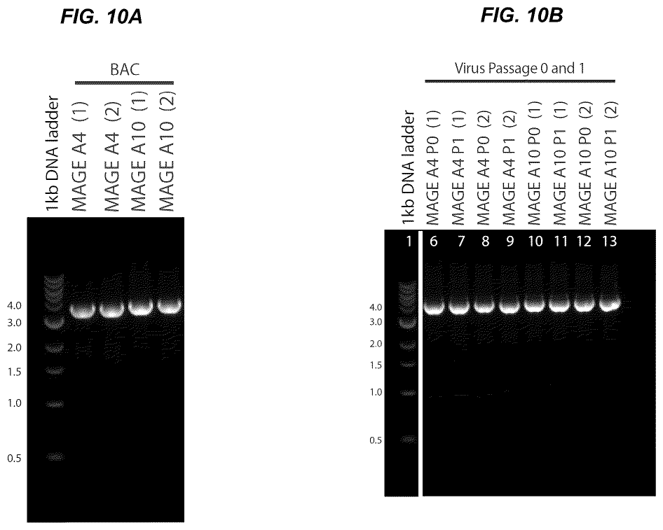

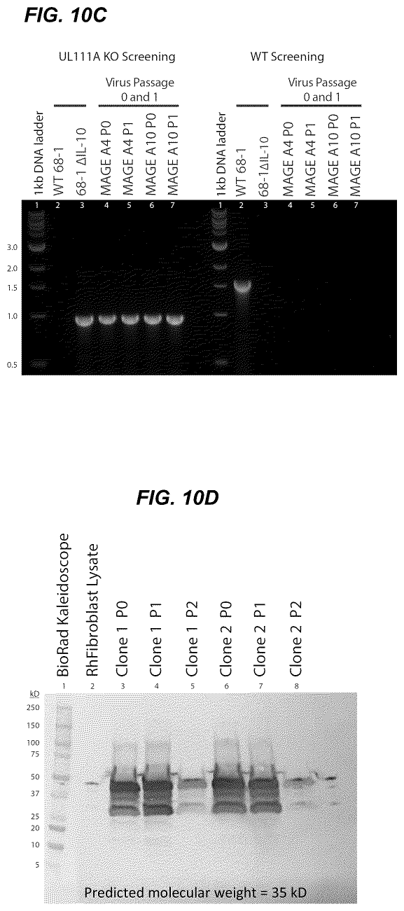

We verified the final RhCMVΔIL-10/MAGEA4 and RhCMVΔIL-10/MAGEA10 vaccines in several ways. Carriage of MAGEA4 or MAGEA10 was confirmed by PCR amplification of a cassette of the correct size in the Rh213/Rh214 region of the viral genome (FIG. 10A). Rescue of replicating vectors from those BACs, and continued carriage of the MAGE genes, was verified by an identical PCR amplification from virions produced in tissue culture after transfection of the BACs (FIG. 10B, “P0”) or after one passage of the replicating vector onto fresh cells (“P1”). Deletion of the first two exons of the viral IL-10 gene and replacement with a Zeocin-resistance cassette was verified by PCR reactions demonstrating both presence of Zeocin^{R} (FIG. 10C, left) and absence of UL111A (FIG. 10C, right). Finally, robust expression of MAGEA4 protein from the RhCMVΔIL-10/MAGEA4 vaccine could be confirmed due to availability of a good antibody for use in Western blotting. Protein expression was detected during serial passage of 2/2 clones of this vaccine vector (FIG. 10D, clones 1 and 2 at P0, P1, and P2).

### VII. Exemplary Embodiments

Exemplary embodiments provided in accordance with the presently disclosed subject matter include, but are not limited to, the claims and the following embodiments:

1. A recombinant polynucleotide comprising a cytomegalovirus (CMV) genome, or a portion thereof, and a nucleic acid sequence encoding an antigen, wherein the CMV genome or portion thereof comprises one or more immunomodulatory mutations, wherein the one or more immunomodulatory mutations comprise a mutation within a nucleic acid sequence encoding a protein that has interleukin-10 (IL-10)-like activity.
2. The recombinant polynucleotide of embodiment 1, wherein the CMV is a CMV that can infect human, non-human primate, or mouse cells.
3. The recombinant polynucleotide of embodiment 1 or 2, wherein the protein that has IL-10-like activity is human CMV IL-10 (HCMVIL-10) or rhesus macaque CMV IL-10 (RhCMVIL-10).
4. The recombinant polynucleotide of any one of embodiments 1 to 3, wherein the nucleotide sequence encoding the antigen is located within the CMV genome or portion thereof.
5. The recombinant polynucleotide of any one of embodiments 1 to 4, wherein the one or more immunomodulatory mutations comprise a substitution, a deletion, and/or an insertion of one or more nucleotides.
6. The recombinant polynucleotide of any one of embodiments 1 to 5, wherein the one or more immunomodulatory mutations are located in a regulatory region and/or a protein coding region of the nucleic acid sequence encoding the protein that has IL-10-like activity.
7. The recombinant polynucleotide of any one of embodiments 1 to 6, wherein the mutation within the nucleic acid sequence encoding the protein that has IL-10-like activity comprises a deletion within the first two exons of the nucleic acid sequence encoding the protein that has IL-10-like activity.
8. The recombinant polynucleotide of any one of embodiments 1 to 7, wherein the mutation within the nucleic acid sequence encoding the protein that has IL-10-like activity reduces or inactivates the activity of the protein having IL-10-like activity.
9. The recombinant polynucleotide of any one of embodiments 1 to 8, wherein the antigen is a non-CMV antigen.
10. The recombinant polynucleotide of any one of embodiments 1 to 9, wherein the antigen is an infectious disease antigen.
11. The recombinant polynucleotide of embodiment 10, wherein the infectious disease antigen is a bacterial, viral, fungal, protozoal, and/or helminthic infectious disease antigen.
12. The recombinant polynucleotide of embodiment 10 or 11, wherein the infectious disease antigen is a viral infectious disease antigen from simian immunodeficiency virus (SIV), human immunodeficiency virus (HIV), hepatitis C virus, herpes simplex virus, Epstein-Barr virus, or a combination thereof.
13. The recombinant polynucleotide of any one of embodiments 10 to 12, wherein the infectious disease antigen comprises an HIV or SIV group-specific antigen (gag) protein.
14. The recombinant polynucleotide of embodiment 10 or 11, wherein the infectious disease antigen is a bacterial infectious disease antigen from Mycobacterium tuberculosis.
15. The recombinant polynucleotide of any one of embodiments 1 to 9, wherein the antigen is a tumor-associated antigen.
16. The recombinant polynucleotide of embodiment 15, wherein the tumor-associated antigen is selected from the group consisting of prostate-specific antigen, melanoma-associated antigen 4 (MAGEA4), melanoma-associated antigen 10 (MAGEA10), NY-ESO-1, a neoantigen, and a combination thereof.
17. The recombinant polynucleotide of any one of embodiments 1 to 16, wherein the one or more immunomodulatory mutations further comprise an insertion of a nucleic acid sequence encoding an immunostimulatory protein.
18. The recombinant polynucleotide of embodiment 17, wherein the immunostimulatory protein is a cytokine.
19. The recombinant polynucleotide of embodiment 18, wherein the cytokine is selected from the group consisting of interleukin-12 (IL-12), interleukin-15 (IL-15), and a combination thereof.
20. The recombinant polynucleotide of any one of embodiments 1 to 19, wherein the CMV is a CMV capable of infecting rhesus macaque cells and wherein the one or more immunomodulatory mutations further comprise a mutation within a region of the CMV genome or portion thereof selected from the group consisting of Rh182, Rh183, Rh184, Rh185, Rh186, Rh187, Rh188, Rh189, and a combination thereof.
21. The recombinant polynucleotide of any one of embodiments 1 to 19, wherein the CMV is a CMV capable of infecting human cells and wherein the one or more immunomodulatory mutations further comprise a mutation within a region of the CMV genome or portion thereof selected from the group consisting of US2, US3, US4, US5, US6, US7, US8, US9, US10, US11, and a combination thereof.
22. The recombinant polynucleotide of any one of embodiments 1 to 21, wherein the one or more immunomodulatory mutations further comprise a mutation within a nucleic acid sequence encoding a protein that inhibits antigen presentation by a major histocompatibility complex (MHC) molecule.
23. The recombinant polynucleotide of any one of embodiments 1 to 22, wherein the CMV genome or portion thereof further comprises a mutation that increases tropism for a target cell.
24. The recombinant polynucleotide of embodiment 23, wherein the target cell is selected from the group consisting of an antigen-presenting cell, a tumor cell, a fibroblast, an epithelial cell, an endothelial cell, and a combination thereof.
25. The recombinant polynucleotide of embodiment 24, wherein the antigen-presenting cell is a dendritic cell.
26. The recombinant polynucleotide of any one of embodiments 23 to 25, wherein the mutation that increases tropism comprises a mutation that modifies a protein, or a portion thereof, that is positioned on the outside of a CMV virion.
27. The recombinant polynucleotide of any one of embodiments 23 to 26, wherein the mutation that increases tropism comprises an insertion of a nucleotide sequence encoding a cellular targeting ligand.
28. The recombinant polynucleotide of embodiment 27, wherein the cellular targeting ligand is selected from the group consisting of an antibody fragment that recognizes a target cell antigen, a ligand that is recognized by a target cell cognate receptor, a viral capsid protein that recognizes a target cell, and a combination thereof.
29. The recombinant polynucleotide of embodiment 27 or 28, wherein the cellular targeting ligand is CD154.
30. The recombinant polynucleotide of any one of embodiments 23 to 29, wherein the CMV is a CMV capable of infecting rhesus macaque cells and wherein the mutation that increases tropism comprises a mutation within a gene selected from the group consisting of Rh13.1, Rh61/Rh60, Rh157.4, Rh157.5, Rh157.6, and a combination thereof.
31. The recombinant polynucleotide of any one of embodiments 23 to 29, wherein the CMV is a CMV capable of infecting human cells and wherein the mutation that increases tropism comprises a mutation within a gene selected from the group consisting of RL13, UL36, UL130, UL128, UL131, and a combination thereof.
32. The recombinant polynucleotide of any one of embodiments 1 to 31, wherein the one or more immunomodulatory mutations further comprise a mutation that increases or decreases the unfolded protein response (UPR).
33. The recombinant polynucleotide of embodiment 32, wherein the mutation that increases or decreases the UPR decreases or increases the expression of Human cytomegalovirus UL50, Rhesus cytomegalovirus Rh81, or Mouse cytomegalovirus M50.
34. The recombinant polynucleotide of any one of embodiments 1 to 33, wherein the polynucleotide further comprises a nucleic acid sequence encoding a selectable marker.
35. The recombinant polynucleotide of embodiment 34, wherein the nucleic acid sequence encoding the selectable marker is located within the CMV genome or portion thereof.
36. The recombinant polynucleotide of embodiment 34 or 35, wherein the nucleic acid sequence encoding the selectable marker comprises a nucleic acid sequence encoding an antibiotic resistance gene and/or a fluorescent protein.
37. The recombinant polynucleotide of any one of embodiments 1 to 36, wherein the recombinant polynucleotide contains one or more regulatory sequences.
38. The recombinant polynucleotide of embodiment 37, wherein the one or more regulatory sequences control the expression of a gene or region within the CMV genome or portion thereof, the antigen-encoding sequence, an immunostimulatory protein-encoding sequence, a selectable marker-encoding sequence, a variant thereof, or a combination thereof.
39. The recombinant polynucleotide of embodiment 37 or 38, wherein the one or more regulatory sequences comprise a CMV early enhancer, a chicken beta-actin gene promoter, a first exon of a chicken beta-actin gene, a first intron of a chicken beta-actin gene, a splice acceptor of a rabbit beta-globin gene, an EM7 promoter, an EF1α promoter, or a combination thereof.
40. A viral particle comprising the recombinant polynucleotide of any one of embodiments 1 to 39.
41. A host cell comprising the recombinant polynucleotide of any one of embodiments 1 to 39, or the viral particle of embodiment 40.
42. A pharmaceutical composition comprising:
(a) the recombinant polynucleotide of any one of embodiments 1 to 39, the viral particle of embodiment 40, or the host cell of embodiment 41; and
(b) a pharmaceutically acceptable carrier.
   43. A method for inducing an immune response against an antigen in a subject, the method comprising administering to the subject a therapeutically effective amount of the pharmaceutical composition of embodiment 42.
   44. The method of embodiment 43, wherein the antigen is an infectious disease antigen or a tumor-associated antigen.
   45. The method of embodiment 44, wherein the infectious disease antigen is a bacterial, viral, fungal, protozoal, and/or helminthic infectious disease antigen.
   46. The method of embodiment 45, wherein the viral infectious disease antigen is from simian immunodeficiency virus (SIV), human immunodeficiency virus (HIV), hepatitis C virus, herpes simplex virus, Epstein-Barr virus, or a combination thereof.
   47. The method of embodiment 45, wherein the bacterial infectious disease antigen is from Mycobacterium tuberculosis.
   48. The method of embodiment 44, wherein the tumor-associated antigen is selected from the group consisting of prostate-specific antigen, melanoma-associated antigen 4 (MAGEA4), melanoma-associated antigen 10 (MAGEA10), NY-ESO-1, a neoantigen, and a combination thereof.
   49. The method of any one of embodiments 43 to 48, wherein the immune response induced in the subject is greater than the immune response that is induced using a recombinant polynucleotide that does not comprise the mutation within the nucleic acid sequence encoding the protein that has IL-10-like activity.
   50. The method of any one of embodiments 43 to 49, wherein inducing the immune response comprises generating antibodies that recognize the antigen.
   51. The method of any one of embodiments 43 to 50, wherein inducing the immune response comprises increasing the expression or activity of interferon-gamma and/or tumor necrosis factor-alpha in the subject.
   52. The method of any one of embodiments 43 to 51, wherein inducing the immune response comprises increasing the number or activation of MHC-E-restricted T cells in the subject.
   53. The method of any one of embodiments 43 to 52, wherein the unfolded protein response (UPR) is increased or decreased in the subject.
   54. The method of any one of embodiments 43 to 53, wherein a sample is obtained from the subject.
   55. The method of embodiment 54, wherein the sample is selected from the group consisting of a blood sample, a tissue sample, a urine sample, a saliva sample, a cerebrospinal fluid (CSF) sample, and a combination thereof.
   56. The method of embodiment 54 or 55, wherein the level of one or more biomarkers is determined in the sample.
   57. The method of embodiment 56, wherein the one or more biomarkers is selected from the group consisting of C-reactive protein, interferon-gamma, IL-4, IL-5, IL-6, IL-10, IL-12, IL-15, tumor necrosis factor-alpha, and a combination thereof.
   58. The method of embodiment 56 or 57, wherein the level of the one or more biomarkers is compared to a reference sample.
   59. The method of embodiment 58, wherein the reference sample is obtained from the subject.
   60. The method of embodiment 58, wherein the reference sample is obtained from a different subject or a population of subjects.
   61. A method for preventing or treating a disease in a subject, the method comprising administering to the subject a therapeutically effective amount of the pharmaceutical composition of embodiment 42.
   62. The method of embodiment 61, wherein the disease is an infectious disease or cancer.
   63. The method of embodiment 62, wherein the infectious disease is a bacterial, viral, fungal, protozoal, and/or helminthic infectious disease.
   64. The method of embodiment 63, wherein the viral infectious disease is caused by a virus selected from the group consisting of simian immunodeficiency virus (SIV), human immunodeficiency virus (HIV), hepatitis C virus, herpes simplex virus, and Epstein-Barr virus.
   65. The method of embodiment 63, wherein the bacterial infectious disease is caused by Mycobacterium tuberculosis.
   66. The method of embodiment 62, wherein the cancer is melanoma, ovarian cancer, or prostate cancer.
   67. The method of any one of embodiments 61 to 66, wherein treating the subject comprises decreasing or eliminating one or more signs or symptoms of the disease.

It is understood that the examples and embodiments described herein are for illustrative purposes only and that various modifications or changes in light thereof will be suggested to persons skilled in the art and are to be included within the spirit and purview of this application and scope of the appended claims. All publications, patents, patent applications, and sequence reference numbers cited herein are hereby incorporated by reference in their entirety for all purposes.

TABLE 1
Informal Sequence Listing

| SEQ | | |
|---|---|---|
| ID | | |
| NO: | Sequence | Description |
| 1 | 5′-TCGGTGCTGTTGTTTAGCCTGGAGAAGGAGACGAGAACGACGAATCGGCG- | Sequence |
| | 3′ | homologous to |
| | | RhCMV and |
| | | flanking EM7- |
| | | Zeocin cassette |
| 2 | 5′-ATCGCTATTTACGTGTCATATCGCGGGGTTATCGAACCTGAAACACTTAC-3′ | Sequence |
| | | homologous to |
| | | RhCMV and |
| | | flanking EM7- |
| | | Zeocin cassette |
| 3 | 5′- | Sequence of EM7- |
| | TGTTGACAATTAATCATCGGCATAGTATATCGGCATAGTATAATACGACAAGG | Zeocin cassette |
| | TGAGGAACTAAACCATGGCCAAGTTGACCAGTGCCGTTCCGGTGCTCACCGCG | retained in |
| | CGCGACGTCGCCGGAGCGGTCGAGTTCTGGACCGACCGGCTCGGGTTCTCCCG | RhCMVΔIL-10 |
| | GGACTTCGTGGAGGACGACTTCGCCGGTGTGGTCCGGGACGACGTGACCCTGT | |
| | TCATCAGCGCGGTCCAGGACCAGGTGGTGCCGGACAACACCCTGGCCTGGGT | |
| | GTGGGTGCGCGGCCTGGACGAGCTGTACGCCGAGTGGTCGGAGGTCGTGTCC | |
| | ACGAACTTCCGGGACGCCTCCGGGCCGGCCATGACCGAGATCGGCGAGCAGC | |
| | CGTGGGGGCGGGAGTTCGCCCTGCGCGACCCGGCCGGCAACTGCGTGCACTTC | |
| | GTGGCCGAGGAGCAGGACTGAGAATTCCC-3′ | |
| 4 | 5′- | Primer sequence |
| | TCGGTGCTGTTGTTTAGCCTGGAGAAGGAGACGAGAACGACGAATCGGCGTG | |
| | TTGACAATTAATCATCGGCATAG-3′ | |
| 5 | 5′- | Primer sequence |
| | ATCGCTATTTACGTGTCATATCGCGGGGTTATCGAACCTGAAACACTTACGGG | |
| | AATTCTCAGTCCTGCTCCTCGG-3′ | |
| 6 | 5′-TGGCGTCTCATTCTCTGTTGCAG-3′ | Primer sequence |
| 7 | 5′-AAGACTGTGACTGACGTCTGGTG-3′ | Primer sequence |
| 8 | 5′- | EF1α promoter |
| | CGTGAGGCTCCGGTGCCCGTCAGTGGGCAGAGCGCACATCGCCCACAGTCCCC | sequence |
| | GAGAAGTTGGGGGGAGGGGTCGGCAATTGAACCGGTGCCTAGAGAAGGTGGC | |
| | GCGGGGTAAACTGGGAAAGTGATGTCGTGTACTGGCTCCGCCTTTTTCCCGAG | |
| | GGTGGGGGAGAACCGTATATAAGTGCAGTAGTCGCCGTGAACGTTCTTTTTCG | |
| | CAACGGGTTTGCCGCCAGAACACAGGTAAGTGCCGTGTGTGGTTCCCGCGGGC | |
| | CTGGCCTCTTTACGGGTTATGGCCCTTGCGTGCCTTGAATTACTTCCACCTGGC | |
| | TGCAGTACGTGATTCTTGATCCCGAGCTTCGGGTTGGAAGTGGGTGGGAGAGT | |
| | TCGAGGCCTTGCGCTTAAGGAGCCCCTTCGCCTCGTGCTTGAGTTGAGGCCTG | |
| | GCCTGGGCGCTGGGGCCGCCGCGTGCGAATCTGGTGGCACCTTCGCGCCTGTC | |
| | TCGCTGCTTTCGATAAGTCTCTAGCCATTTAAAATTTTTGATGACCTGCTGCGA | |
| | CGCTTTTTTTCTGGCAAGATAGTCTTGTAAATGCGGGCCAAGATCTGCACACT | |
| | GGTATTTCGGTTTTTGGGGCCGCGGGCGGCGACGGGGCCCGTGCGTCCCAGCG | |
| | CACATGTTCGGCGAGGCGGGGCCTGCGAGCGCGGCCACCGAGAATCGGACGG | |
| | GGGTAGTCTCAAGCTGGCCGGCCTGCTCTGGTGCCTGGCCTCGCGCCGCCGTG | |
| | TATCGCCCCGCCCTGGGCGGCAAGGCTGGCCCGGTCGGCACCAGTTGCGTGAG | |
| | CGGAAAGATGGCCGCTTCCCGGCCCTGCTGCAGGGAGCTCAAAATGGAGGAC | |
| | GCGGCGCTCGGGAGAGCGGGCGGGTGAGTCACCCACACAAAGGAAAAGGGC | |
| | CTTTCCGTCCTCAGCCGTCGCTTCATGTGACTCCACGGAGTACCGGGCGCCGT | |
| | CCAGGCACCTCGATTAGTTCTCGAGCTTTTGGAGTACGTCGTCTTTAGGTTGGG | |
| | GGGAGGGGTTTTATGCGATGGAGTTTCCCCACACTGAGTGGGTGGAGACTGA | |
| | AGTTAGGCCAGCTTGGCACTTGATGTAATTCTCCTTGGAATTTGCCCTTTTTGA | |
| | GTTTGGATCTTGGTTCATTCTCAAGCCTCAGACAGTGGTTCAAAGTTTTTTTCT | |
| | TCCATTTCAGGTGTCGTGA-3′ | |
| 9 | 5′- | Sequence of |
| | ATGGGCGTGAGAAACTCCGTCTTGTCAGGGAAGAAAGCAGATGAATTAGAAA | codon-optimized |
| | AAATTAGGCTACGACCCAACGGAAAGAAAAAGTACATGTTGAAGCATGTAGT | SIV gag-Flag |
| | ATGGGCAGCAAATGAATTAGATAGATTTGGATTAGCAGAAAGCCTGTTGGAG | fusion protein |
| | AACAAAGAAGGATGTCAAAAAATACTTTCGGTCTTAGCTCCATTAGTGCCAAC | |
| | AGGCTCAGAAAATTTAAAAAGCCTTTATAATACTGTCTGCGTCATCTGGTGCA | |
| | TTCACGCAGAAGAGAAAGTGAAACACACTGAGGAAGCAAAACAGATAGTGC | |
| | AGAGACACCTAGTGGTGGAAACAGGAACCACCGAAACCATGCCGAAGACCTC | |
| | TCGACCAACAGCACCATCTAGCGGCAGAGGAGGAAACTACCCAGTACAGCAG | |
| | ATCGGTGGCAACTACGTCCACCTGCCACTGTCCCCGAGAACCCTGAACGCTTG | |
| | GGTCAAGCTGATCGAGGAGAAGAAGTTCGGAGCAGAAGTAGTGCCAGGATTC | |
| | CAGGCACTGTCAGAAGGTTGCACCCCCTACGACATCAACCAGATGCTGAACTG | |
| | CGTTGGAGACCATCAGGCGGCTATGCAGATCATCCGTGACATCATCAACGAG | |
| | GAGGCTGCAGATTGGGACTTGCAGCACCCACAACCAGCTCCACAACAAGGAC | |
| | AACTTAGGGAGCCGTCAGGATCAGACATCGCAGGAACCACCTCCTCAGTTGA | |
| | CGAACAGATCCAGTGGATGTACCGTCAGCAGAACCCGATCCCAGTAGGCAAC | |
| | ATCTACCGTCGATGGATCCAGCTGGGTCTGCAGAAATGCGTCCGTATGTACAA | |
| | CCCGACCAACATTCTAGATGTAAAACAAGGGCCAAAAGAGCCATTTCAGAGC | |
| | TATGTAGACAGGTTCTACAAAAGTTTAAGAGCAGAACAGACAGATGCAGCAG | |
| | TAAAGAATTGGATGACTCAAACACTGCTGATTCAAAATGCTAACCCAGATTGC | |
| | AAGCTAGTGCTGAAGGGGCTGGGTGTGAATCCCACCCTAGAAGAAATGCTGA | |
| | CGGCTTGTCAAGGAGTAGGGGGGCCGGGACAGAAGGCTAGATTAATGGCAGA | |
| | AGCCCTGAAAGAGGCCCTCGCACCAGTGCCAATCCCTTTTGCAGCAGCCCAAC | |
| | AGAGGGGACCAAGAAAGCCAATTAAGTGTTGGAATTGTGGGAAAGAGGGAC | |
| | ACTCTGCAAGGCAATGCAGAGCCCCAAGAAGACAGGGATGCTGGAAATGTGG | |
| | AAAAATGGACCATGTTATGGCCAAATGCCCAGACAGACAGGCGGGTTTTTTA | |
| | GGCCTTGGTCCATGGGGAAAGAAGCCCCGCAATTTCCCCATGGCTCAAGTGCA | |
| | TCAGGGGCTGATGCCAACTGCTCCCCCAGAGGACCCAGCTGTGGATCTGCTAA | |
| | AGAACTACATGCAGTTGGGCAAGCAGCAGAGAGAAAAGCAGAGAGAAAGCA | |
| | GAGAGAAGCCTTACAAGGAGGTGACAGAGGATTTGCTGCACCTCAATTCTCTC | |
| | TTTGGAGGAGACCAGGACTACAAAGACGATGACGACAAGTAG-3′ | |
| 10 | 5′- | SV40 |
| | TAACTTGTTTATTGCAGCTTATAATGGTTACAAATAAAGCAATAGCATCACAA | polyadenylation |
| | ATTTCACAAATAAAGCATTTTTTTCACTGCATTCTAGTTGTGGTTTGTGGAAAC | site sequence |
| | TCATCAATGTATCTTATCATGTC -3′ | |
| 11 | 5′- | Viral IL-10-like |
| | ATGCGGAGGAGGAGGAGGTCTTTCGGCATCATCGTCGCCGGCGCTATCGGAA | sequence from |
| | CACTACTCATGATGGCGGTGGTCGTGCTTTCAGCCCATGACCATGAACACAAA | RhCMV strain 68- |
| | GAAGTACCACCGGCCTGTGACCCCGTTCACGGTAACTTGGCAGGCATCTTCAA | 1 |
| | GGAGTTGCGGGCGACCTACGCTTCCATTAGAGAAGGTTTGGTATGTTAGGCAA | |
| | CGCAGTTCTCGGATGTCAGTCCGGATCGGAGGAGTCACAGTCTGTCATGTGAT | |
| | GATATATTGCTTAATTTTTGTTTTGCAGCAAAAGAAGGACACGGTGTACTACA | |
| | CATCGCTGTTCAATGACCGCGTGCTCCATGAAATGCTGAGTCCTATGGGCTGT | |
| | CGCGTGACCAATGAACTCATGGAACATTATTTAGATGGTGTTCTGCCTCGAGC | |
| | AAGTCATTTAGACTACGATAATAGCACTCTGAATGGCTTACATGTGTTTGCTTC | |
| | ATCCATGCAGGCGCTGTATCAGCACATGTTAAAGTGTGTAAGTGTTTCAGGTT | |
| | CGATAACCCCGCGATATGACACGTAAATAGCGATATCGTGGCACCAGACGTC | |
| | AGTCACAGTCTTCCCCGGTCGAGACGCATCTTATATCGCGATATATCGCGGAT | |
| | TATCGCAGTATGTAGCGATATATCGTGTCAAAGCACTCCGAACGACATTCTGA | |
| | TGACGGCTATCGCCTTATGTCGCGGTATATCGCGGAATATCGCAGTATATCGC | |
| | GGTTATGTCGCGACATAACCGTCATGTCGCGACTATCGCCGCATATCGCCACT | |
| | ATCGCGACTTGGCACCGTGCCAACGATAGTCGACCTTAGGGTGGTCGTGTGGT | |
| | GGTGGGGGGCTGCTTGCGGTTTGCAAACCGGAGAGGTAGCACACGCTGATTG | |
| | TCGGTTTGGAAGCGTTGTTTACACATGTCTTTGTCTTGGCAGCCCGCGTTGGCA | |
| | TGTACTGGCAAAACGCCAGCTTGGATGTACTTCTTGGAGGTGGAACACAAGGT | |
| | CAGTTAAGGTTGCCAGGTAGGTTAAAACGCAGAAACCATTGTTCTACCGGTTT | |
| | CCTAAAACGCCGTTCAACGTGTTTTGCAGCTCAACCCCTGGAGGGGCACGGCA | |
| | AAAGCCGCGGCCGAGGCTGACCTTTTGCTGAACTACTTGGAAACGTTCCTGCT | |
| | GCAGTTCTGA-3′ | |
| 12 | 5′- | Viral IL-10-like |
| | ATGCGGAGGAGGAGGAGGTCTTTCGGCATCATCGTCGCCGGCGCTATCGGAA | sequence from |
| | CACTACTCATGATGGCGGTGGTCGTGCTTTCAGCCCATGAACACAAAGAAGTA | Cercopithecine |
| | CCACCGGCCTGTGACCCCGTTCACGGTAACTTGGCAGGCATCTTCAAGGAGTT | herpesvirus 8 |
| | GCGGGCGACCTACGCTTCCATTAGAGAAGGTTTGGTATGTTAGGCAACGCAGT | isolate CMV |
| | TCTCGGATGTCAGTCCGGATCGGAGGAGTCACAGTCTGTCATGTGATGATATA | 180.92 |
| | TTGCTTCATTTTTGTTTTGCAGCAAAAGAAGGACACGGTGTACTACACATCGC | |
| | TGTTCAATGACCGCGTGCTCCATGAGATGCTGAGTCCTATGGGCTGTCGCGTG | |
| | ACCAACGAACTCATGGAACATTATTTAGATGGTGTTCTGCCTCGAGCAAGTCA | |
| | TTTAGACTACGATAATAGCACTCTGAATGGCTTACATGTGTTTGCTTCATCCAT | |
| | GCAGGCGCTGTATCAGCACATGTTAAAGTGTGTAAGTGTTTCAGGTTCGATAA | |
| | CCCCGCGATATGACACGTAAATAGCGATATCGTGGCACCAGACGTCAGTCAC | |
| | AGTCTTCCCCGGTCGAGACGCATCTTATATCGCGATATATCGCGGTTTATCGC | |
| | AGTATGTCGCGATATATCGCTCCAAAACACTCCGGATGACTTTCTATCGCCGA | |
| | ATATCACCTCATATCGTCTTATATCGCGGTGTATCGCGGGTTATCGTCATATAT | |
| | CGCGGTTATGTCGCGACATAACCGTCATGTCGCGACTATCGCCGCATATCGCC | |
| | ACTATCGCGACTTGGCACGGTGCCAACAATAGTTGCCTCTAGGGTGGTCGTGT | |
| | GGTGGTAGGGGGCTGCTTACGGTTTGCAAACCGGAGAGGTCGCACACGCTGA | |
| | TTGTCGGTTTGGAAGCGTTGTTTACACATGTCTTTGTCTTGGCAGCCCGCGTTG | |
| | GCATGTACTGGCAAAACGCCAGCTTGGATGTACTTCTTGGAGGTGGAACACAA | |
| | GGTCAGTTAAGGTTGCCAGGTAGGTTAAAACGCAGAAACCATTGTTCTACCGG | |
| | TTTCCTAAAACGCCGTTCAACGTGTTTTGCAGCTCAACCCCTGGAGGGGCACG | |
| | GCAAAAGCCGCGGCCGAGGCTGACCTTTTGCTGAACTACTTGGAAACGTTCCT | |
| | GCTGCAGTTCTGA-3′ | |
| 13 | MHSSALLCCLVLLTGVRASPGQGTQSENSCTRFPGNLPHMLRDLRDAFSRVKTFF | Rhesus macaque |
| | QMKDQLDNILLKESLLEDFKGYLGCQALSEMIQFYLEEVMPQAENHDPDIKEHVN | IL-10 amino acid |
| | SLGENLKTLRLRLRRCHRFLPCENKSKAVEQVKNAFSKLQEKGVYKAMSEFDIFI | sequence |
| | NYIEAYMTMKIQN | |
| 14 | MHSSALLCCLVLLTGVRASPGQGTQSENSCTHFPGNLPNMLRDLRDAFSRVKTFF | Human IL-10 |
| | QMKDQLDNLLLKESLLEDFKGYLGCQALSEMIQFYLEEVMPQAENQDPDIKAHV | |
| | NSLGENLKTLRLRLRRCHRFLPCENKSKAVEQVKNAFNKLQEKGIYKAMSEFDIFI | |
| | NYIEAYMTMKIRN | |

1. A recombinant polynucleotide comprising a cytomegalovirus (CMV) genome, or a portion thereof, and a nucleic acid sequence encoding an antigen, wherein the CMV genome or portion thereof has a substantially reduced expression level of a protein that has interleukin-10 (IL-10)-like activity.

2. (canceled)

3. The recombinant polynucleotide of claim 1, wherein the protein that has IL-10-like activity is human CMV IL-10 (HCMVIL-10) or rhesus macaque CMV IL-10 (RhCMVIL-10).

4. The recombinant polynucleotide of claim 1, wherein the nucleotide sequence encoding the antigen is located within the CMV genome or portion thereof.

5. (canceled)

6. The recombinant polynucleotide of claim 68, wherein the one or more immunomodulatory mutations are located in a regulatory region and/or a protein coding region of the nucleic acid sequence encoding the protein that has IL-10-like activity.

7. The recombinant polynucleotide of claim 68, wherein the mutation within the nucleic acid sequence encoding the protein that has IL-10-like activity comprises a deletion within the first two exons of the nucleic acid sequence encoding the protein that has IL-10-like activity.

8. (canceled)

9. The recombinant polynucleotide of claim 1, wherein the antigen is a non-CMV antigen, an infectious disease antigen, or a tumor-associated antigen.

10. (canceled)

11. (canceled)

12. The recombinant polynucleotide of claim 9, wherein the infectious disease antigen is a viral infectious disease antigen from simian immunodeficiency virus (SIV), human immunodeficiency virus (HIV), hepatitis C virus, herpes simplex virus, Epstein-Barr virus, or a combination thereof.

13. The recombinant polynucleotide of claim 9, wherein the infectious disease antigen comprises an HIV or SIV group-specific antigen (gag) protein.

14. (canceled)

15. (canceled)

16. The recombinant polynucleotide of claim 9, wherein the tumor-associated antigen is selected from the group consisting of prostate-specific antigen, melanoma-associated antigen 4 (MAGEA4), melanoma-associated antigen 10 (MAGEA10), NY-ESO-1, a neoantigen, and a combination thereof.

17. The recombinant polynucleotide of claim 68, wherein the one or more immunomodulatory mutations further comprise an insertion of a nucleic acid sequence encoding an immunostimulatory protein.

18. The recombinant polynucleotide of claim 17, wherein the immunostimulatory protein is a cytokine, wherein the cytokine is selected from the group consisting of interleukin-12 (IL-12), interleukin-15 (IL-15), and a combination thereof.

19. (canceled)

20. The recombinant polynucleotide of claim 68, wherein the CMV is a CMV capable of infecting rhesus macaque cells and wherein the one or more immunomodulatory mutations further comprise a mutation within a region of the CMV genome or portion thereof selected from the group consisting of Rh182, Rh183, Rh184, Rh185, Rh186, Rh187, Rh188, Rh189, and a combination thereof.

21. The recombinant polynucleotide of claim 68, wherein the CMV is a CMV capable of infecting human cells and wherein the one or more immunomodulatory mutations further comprise a mutation within a region of the CMV genome or portion thereof selected from the group consisting of US2, US3, US4, US5, US6, US7, US8, US9, US10, US11, and a combination thereof.

22. The recombinant polynucleotide of claim 68, wherein the one or more immunomodulatory mutations further comprise a mutation within a nucleic acid sequence encoding a protein that inhibits antigen presentation by a major histocompatibility complex (MEW) molecule.

23. The recombinant polynucleotide of claim 1, wherein the CMV genome or portion thereof further comprises a mutation that increases tropism for a target cell, wherein the target cell is selected from the group consisting of an antigen-presenting cell, a tumor cell, a fibroblast, an epithelial cell, an endothelial cell, and a combination thereof.

24. (canceled)

25. (canceled)

26. (canceled)

27. The recombinant polynucleotide of claim 23, wherein the mutation that increases tropism comprises an insertion of a nucleotide sequence encoding a cellular targeting ligand, wherein the cellular targeting ligand is selected from the group consisting of an antibody fragment that recognizes a target cell antigen, a ligand that is recognized by a target cell cognate receptor, a viral capsid protein that recognizes a target cell, and a combination thereof.

28. (canceled)

29. The recombinant polynucleotide of claim 27, wherein the cellular targeting ligand is CD154.

30. (canceled)

31. The recombinant polynucleotide of claim 23, wherein the CMV is a CMV capable of infecting human cells and wherein the mutation that increases tropism comprises a mutation within a gene selected from the group consisting of RL13, UL36, UL130, UL128, UL131, and a combination thereof.

32. The recombinant polynucleotide of claim 68, wherein the one or more immunomodulatory mutations further comprise a mutation that increases or decreases the unfolded protein response (UPR).

33. The recombinant polynucleotide of claim 32, wherein the mutation that increases or decreases the UPR decreases or increases the expression of Human cytomegalovirus UL50, Rhesus cytomegalovirus Rh81, or Mouse cytomegalovirus M50.

34-67. (canceled)

68. The recombinant polynucleotide of claim 1, wherein the CMV genome or portion thereof comprises one or more immunomodulatory mutations, wherein the one or more immunomodulatory mutations comprise a mutation within a nucleic acid sequence encoding the protein that has IL-10-like activity.


SEQUENCE LISTING (15 sequences)

SEQ ID NO: 1 - DNA, 50 bp, Rhesus cytomegalovirus
  source 1..50: mol_type = genomic DNA
tcggtgctgttgtttagcctggagaaggagacgagaacgacgaatcggcg

SEQ ID NO: 2 - DNA, 50 bp, Rhesus cytomegalovirus
  source 1..50: mol_type = genomic DNA
atcgctatttacgtgtcatatcgcggggttatcgaacctgaaacacttac

SEQ ID NO: 3 - DNA, 450 bp, synthetic construct
  misc_feature 1..450: note = Description of Artificial Sequence: Synthetic polynucleotide
  source 1..450: mol_type = other DNA
tgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgagaattccc

SEQ ID NO: 4 - DNA, 75 bp, synthetic construct
  misc_feature 1..75: note = Description of Artificial Sequence: Synthetic primer
  source 1..75: mol_type = other DNA
tcggtgctgttgtttagcctggagaaggagacgagaacgacgaatcggcgtgttgacaattaatcatcggcatag

SEQ ID NO: 5 - DNA, 75 bp, synthetic construct
  misc_feature 1..75: note = Description of Artificial Sequence: Synthetic primer
  source 1..75: mol_type = other DNA
atcgctatttacgtgtcatatcgcggggttatcgaacctgaaacacttacgggaattctcagtcctgctcctcgg

SEQ ID NO: 6 - DNA, 23 bp, synthetic construct
  misc_feature 1..23: note = Description of Artificial Sequence: Synthetic primer
  source 1..23: mol_type = other DNA
tggcgtctcattctctgttgcag

SEQ ID NO: 7 - DNA, 23 bp, synthetic construct
  misc_feature 1..23: note = Description of Artificial Sequence: Synthetic primer
  source 1..23: mol_type = other DNA
aagactgtgactgacgtctggtg

SEQ ID NO: 8 - DNA, 1184 bp, unidentified
  misc_feature 1..1184: note = Description of Unknown Sequence: Unknown EF1-alpha promoter sequence
  source 1..1184: mol_type = other DNA
cgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 9 - DNA, 1557 bp, synthetic construct
  misc_feature 1..1557: note = Description of Artificial Sequence: Synthetic polynucleotide
  source 1..1557: mol_type = other DNA
atgggcgtgagaaactccgtcttgtcagggaagaaagcagatgaattagaaaaaattaggctacgacccaacggaaagaaaaagtacatgttgaagcatgtagtatgggcagcaaatgaattagatagatttggattagcagaaagcctgttggagaacaaagaaggatgtcaaaaaatactttcggtcttagctccattagtgccaacaggctcagaaaatttaaaaagcctttataatactgtctgcgtcatctggtgcattcacgcagaagagaaagtgaaacacactgaggaagcaaaacagatagtgcagagacacctagtggtggaaacaggaaccaccgaaaccatgccgaagacctctcgaccaacagcaccatctagcggcagaggaggaaactacccagtacagcagatcggtggcaactacgtccacctgccactgtccccgagaaccctgaacgcttgggtcaagctgatcgaggagaagaagttcggagcagaagtagtgccaggattccaggcactgtcagaaggttgcaccccctacgacatcaaccagatgctgaactgcgttggagaccatcaggcggctatgcagatcatccgtgacatcatcaacgaggaggctgcagattgggacttgcagcacccacaaccagctccacaacaaggacaacttagggagccgtcaggatcagacatcgcaggaaccacctcctcagttgacgaacagatccagtggatgtaccgtcagcagaacccgatcccagtaggcaacatctaccgtcgatggatccagctgggtctgcagaaatgcgtccgtatgtacaacccgaccaacattctagatgtaaaacaagggccaaaagagccatttcagagctatgtagacaggttctacaaaagtttaagagcagaacagacagatgcagcagtaaagaattggatgactcaaacactgctgattcaaaatgctaacccagattgcaagctagtgctgaaggggctgggtgtgaatcccaccctagaagaaatgctgacggcttgtcaaggagtaggggggccgggacagaaggctagattaatggcagaagccctgaaagaggccctcgcaccagtgccaatcccttttgcagcagcccaacagaggggaccaagaaagccaattaagtgttggaattgtgggaaagagggacactctgcaaggcaatgcagagccccaagaagacagggatgctggaaatgtggaaaaatggaccatgttatggccaaatgcccagacagacaggcgggttttttaggccttggtccatggggaaagaagccccgcaatttccccatggctcaagtgcatcaggggctgatgccaactgctcccccagaggacccagctgtggatctgctaaagaactacatgcagttgggcaagcagcagagagaaaagcagagagaaagcagagagaagccttacaaggaggtgacagaggatttgctgcacctcaattctctctttggaggagaccaggactacaaagacgatgacgacaagtag

SEQ ID NO: 10 - DNA, 130 bp, Simian virus 40
  source 1..130: mol_type = genomic DNA
taacttgtttattgcagcttataatggttacaaataaagcaatagcatcacaaatttcacaaataaagcatttttttcactgcattctagttgtggtttgtggaaactcatcaatgtatcttatcatgtc

SEQ ID NO: 11 - DNA, 1175 bp, Rhesus cytomegalovirus
  source 1..1175: mol_type = genomic DNA
atgcggaggaggaggaggtctttcggcatcatcgtcgccggcgctatcggaacactactcatgatggcggtggtcgtgctttcagcccatgaccatgaacacaaagaagtaccaccggcctgtgaccccgttcacggtaacttggcaggcatcttcaaggagttgcgggcgacctacgcttccattagagaaggtttggtatgttaggcaacgcagttctcggatgtcagtccggatcggaggagtcacagtctgtcatgtgatgatatattgcttaatttttgttttgcagcaaaagaaggacacggtgtactacacatcgctgttcaatgaccgcgtgctccatgaaatgctgagtcctatgggctgtcgcgtgaccaatgaactcatggaacattatttagatggtgttctgcctcgagcaagtcatttagactacgataatagcactctgaatggcttacatgtgtttgcttcatccatgcaggcgctgtatcagcacatgttaaagtgtgtaagtgtttcaggttcgataaccccgcgatatgacacgtaaatagcgatatcgtggcaccagacgtcagtcacagtcttccccggtcgagacgcatcttatatcgcgatatatcgcggattatcgcagtatgtagcgatatatcgtgtcaaagcactccgaacgacattctgatgacggctatcgccttatgtcgcggtatatcgcggaatatcgcagtatatcgcggttatgtcgcgacataaccgtcatgtcgcgactatcgccgcatatcgccactatcgcgacttggcaccgtgccaacgatagtcgaccttagggtggtcgtgtggtggtggggggctgcttgcggtttgcaaaccggagaggtagcacacgctgattgtcggtttggaagcgttgtttacacatgtctttgtcttggcagcccgcgttggcatgtactggcaaaacgccagcttggatgtacttcttggaggtggaacacaaggtcagttaaggttgccaggtaggttaaaacgcagaaaccattgttctaccggtttcctaaaacgccgttcaacgtgttttgcagctcaacccctggaggggcacggcaaaagccgcggccgaggctgaccttttgctgaactacttggaaacgttcctgctgcagttctga

SEQ ID NO: 12 - DNA, 1179 bp, Cercopithecine herpesvirus 8
  source 1..1179: mol_type = genomic DNA
atgcggaggaggaggaggtctttcggcatcatcgtcgccggcgctatcggaacactactcatgatggcggtggtcgtgctttcagcccatgaacacaaagaagtaccaccggcctgtgaccccgttcacggtaacttggcaggcatcttcaaggagttgcgggcgacctacgcttccattagagaaggtttggtatgttaggcaacgcagttctcggatgtcagtccggatcggaggagtcacagtctgtcatgtgatgatatattgcttcatttttgttttgcagcaaaagaaggacacggtgtactacacatcgctgttcaatgaccgcgtgctccatgagatgctgagtcctatgggctgtcgcgtgaccaacgaactcatggaacattatttagatggtgttctgcctcgagcaagtcatttagactacgataatagcactctgaatggcttacatgtgtttgcttcatccatgcaggcgctgtatcagcacatgttaaagtgtgtaagtgtttcaggttcgataaccccgcgatatgacacgtaaatagcgatatcgtggcaccagacgtcagtcacagtcttccccggtcgagacgcatcttatatcgcgatatatcgcggtttatcgcagtatgtcgcgatatatcgctccaaaacactccggatgactttctatcgccgaatatcacctcatatcgtcttatatcgcggtgtatcgcgggttatcgtcatatatcgcggttatgtcgcgacataaccgtcatgtcgcgactatcgccgcatatcgccactatcgcgacttggcacggtgccaacaatagttgcctctagggtggtcgtgtggtggtagggggctgcttacggtttgcaaaccggagaggtcgcacacgctgattgtcggtttggaagcgttgtttacacatgtctttgtcttggcagcccgcgttggcatgtactggcaaaacgccagcttggatgtacttcttggaggtggaacacaaggtcagttaaggttgccaggtaggttaaaacgcagaaaccattgttctaccggtttcctaaaacgccgttcaacgtgttttgcagctcaacccctggaggggcacggcaaaagccgcggccgaggctgaccttttgctgaactacttggaaacgttcctgctgcagttctga

SEQ ID NO: 13 - protein, 178 aa, Macaca mulatta
MHSSALLCCLVLLTGVRASPGQGTQSENSCTRFPGNLPHMLRDLRDAFSRVKTFFQMKDQLDNILLKESLLEDFKGYLGCQALSEMIQFYLEEVMPQAENHDPDIKEHVNSLGENLKTLRLRLRRCHRFLPCENKSKAVEQVKNAFSKLQEKGVYKAMSEFDIFINYIEAYMTMKIQN

SEQ ID NO: 14 - protein, 178 aa, Homo sapiens
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 15 - protein, 10 aa, synthetic construct
  REGION 1..10: note = Description of Artificial Sequence: Synthetic peptide
  REGION 1..10: note = MISC_FEATURE - This sequence may encompass 6-10 residues
HHHHHHHHHH